(19) United States
(12) Patent Application Publication
(10) Pub. No.: US 2005/0025718 A1
(43) Pub. Date: Feb. 3, 2005

(54) Medicaments for inhalation comprising an anticholinergic and a betamimetic

(75) Inventors: Christopher John Montague Meade, Maselheim (DE); Michel Pairet, Biberach (DE); Michael P. Pieper, Biberach (DE); Ingo Konetzki, Warthausen (DE)

Correspondence Address: BOEHRINGER INGELHEIM CORPORATION, 900 RIDGEBURY ROAD, P. O. BOX 368, RIDGEFIELD, CT, 06877, US

(73) Assignee: Boehringer Ingelheim International GmbH, Ingelheim (DE)

(21) Appl. No.: 10/891,564

(22) Filed: Jul. 15, 2004

Related U.S. Application Data
(60) Provisional application No. 60/508,124, filed on Oct. 2, 2003.

(30) Foreign Application Priority Data
Jul. 31, 2003 (EP) EP 03 017 349

(51) Int. Cl. A61L 9/04; A61K 9/14; A61K 31/47; A61K 31/4745

(52) U.S. Cl. 424/46; 514/291; 514/312

(57) ABSTRACT

A pharmaceutical composition comprising an anticholinergic and a betamimetic of formula 2 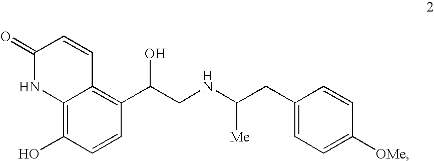 optionally together with a pharmaceutically acceptable excipient, the anticholinergic and the betamimetic optionally in the form of their enantiomers, mixtures of their enantiomers, their racemates, their solvates, or their hydrates, processes for preparing them, and their use in the treatment of asthma, COPD, or other inflammatory or obstructive respiratory complaints.

### RELATED APPLICATIONS

This application claims benefit of U.S. Ser. No. 60/508,124, filed Oct. 2, 2003, and claims priority to European Application No. 03 017 349.6, filed Jul. 31, 2003, each of which is hereby incorporated by reference in its entirety.

### FIELD OF THE INVENTION

The present invention relates to novel pharmaceutical compositions comprising one or more, preferably one, anticholinergic 1 and a betamimetic of formula 2 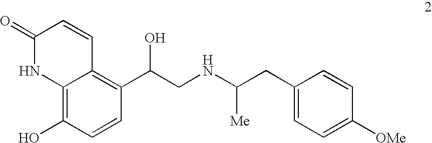
processes for preparing them and their use in the treatment of respiratory complaints.

### DESCRIPTION OF THE FIGURE

FIG. 1 shows an inhaler that may be used for administering the pharmaceutical combination according to the invention in inhalettes.

### DESCRIPTION OF THE INVENTION

The present invention relates to novel pharmaceutical compositions comprising one or more, preferably one, anticholinergic 1 and a betamimetic of formula 2 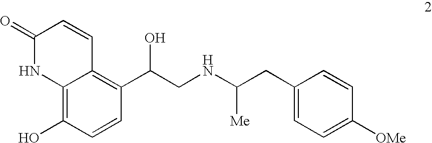
optionally in the form of its diastereomers, mixtures of its diastereomers, racemates, or physiologically acceptable acid addition salts thereof, and optionally in form of the hydrates or solvates thereof, and optionally together with a pharmaceutically acceptable excipient.

Examples of pharmacologically acceptable acid addition salts of the betamimetic 2 according to the invention are the pharmaceutically acceptable salts which are selected from among the salts of hydrochloric acid, hydrobromic acid, sulfuric acid, phosphoric acid, methanesulfonic acid, acetic acid, fumaric acid, succinic acid, lactic acid, citric acid, tartaric acid, 1-hydroxy-2-naphthalenecarboxylic acid, 4-phenylcinnamic acid, or maleic acid. If desired, mixtures of the abovementioned acids may also be used to prepare the salts of 2.

According to the invention, the salts of 2 selected from among the hydrochloride, hydrobromide, sulfate, phosphate, fumarate, methanesulfonate, maleate, and xinafoate are preferred. Particularly preferred is the hydrochloric acid salt of 2.

In the pharmaceutical compositions according to the invention, the compound 2 may be present in the form of its racemates, enantiomers, or mixtures thereof. The separation of the enantiomers from the racemates may be carried out using methods known in the art (e.g., by chromatography on chiral phases, etc.). If the compounds 2 are used in the form of their enantiomers, it is particularly preferable to use the enantiomers possessing the R-configuration at the C—OH group.

Of particular interest within the scope of the instant invention is the R,R-enantiomer of formula 2-en

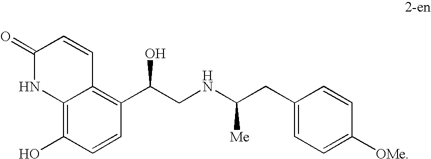

Within the scope of the present invention the betamimetic 2 may possibly also be referred to as sympathomimetic or beta₂-agonist (β₂-agonist). All these terms are to be regarded as interchangeable for the purposes of the present invention.

Within the scope of the present invention the anticholinergic agents 1 are in a preferred embodiment salts selected from among tiotropium salts, oxitropium salts, flutropium salts, ipratropium salts, glycopyrronium salts, and trospium salts. In the abovementioned salts the cations tiotropium, oxitropium, flutropium, ipratropium, glycopyrronium and trospium are the pharmacologically active components. Within the scope of the present patent application, an explicit reference to the above cations is indicated by the use of the symbol 1′. Any reference to the aforementioned salts 1 naturally also includes a reference to the ingredients 1′ (tiotropium, oxitropium, flutropium, ipratropium, glycopyrronium, or trospium). By the salts 1 which may be used within the scope of the present invention are meant the compounds which contain, in addition to tiotropium, oxitropium, flutropium, ipratropium, glycopyrronium, or trospium as counter-ion (anion), chloride, bromide, iodide, sulfate, phosphate, methanesulfonate, nitrate, maleate, acetate, citrate, fumarate, tartrate, oxalate, succinate, benzoate, or p-toluenesulfonate, wherein chloride, bromide, iodide, sulfate, methanesulfonate, or p-toluenesulfonate are preferred. Within the scope of the present invention, the methanesulfonate, chloride, bromide, and iodide are preferred of all the salts 1. If trospium salts are used, the chloride is of particular importance. From the other salts mentioned hereinbefore the methanesulfonate and bromide are of particular importance. Of particular importance according to the invention are salts 1 selected from among tiotropium salts, oxitropium salts, and ipratropium salts. Of outstanding importance according to the invention are salts 1 selected from among tiotropium bromide, oxitropium bromide, and ipratropium bromide. Tiotropium bromide is particularly preferred. The aforementioned salts may be optionally present in form of their solvates or hydrates, preferably in form of their hydrates. If tiotropium bromide is used, it is preferably present in form of its crystalline tiotropium bromide monohydrate as disclosed in WO 02/30928 (corresponding to U.S. Patent App. Pub. No. 2003/0171586, which is hereby incorporated by reference). In case tiotropium bromide is used in anhydrous form, it is preferably present in form of the crystalline tiotropium bromide anhydrate disclosed in WO 03/000265 (corresponding to U.S. Patent App. Pub. No. 2003/0087927, which is hereby incorporated by reference).

Optionally the anticholinergic agents mentioned hereinbefore possess chiral carbon centers. In this case, the pharmaceutical combinations according to the invention may contain the anticholinergic agents in form of their enantiomers, mixtures of enantiomers, or racemates. Preferably chiral anticholinergics are present in form of one of their pure enantiomers.

Within the scope of the present invention the anticholinergic agents 1 are in another preferred embodiment selected from the salts of LAS 34273, having the formula 1a 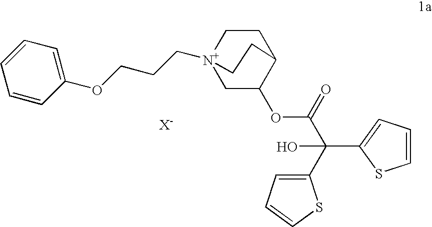
wherein X⁻ denotes an anion with a single negative charge, preferably an anion selected from the group consisting of fluoride, chloride, bromide, iodide, sulfate, phosphate, methanesulfonate, nitrate, maleate, acetate, citrate, fumarate, tartrate, oxalate, succinate, benzoate, and p-toluenesulfonate, optionally in the form of the racemates, the enantiomers, and the hydrates thereof.

Preferably, the salts of formula 1a are used wherein X⁻ denotes an anion with a single negative charge selected from among the fluoride, chloride, bromide, 4-toluenesulfonate, and methanesulfonate, preferably bromide, optionally in the form of the racemates, the enantiomers, and the hydrates thereof.

Most preferably, the salts of formula 1a are used wherein X⁻ denotes an anion with a single negative charge selected from among the chloride, bromide, and methanesulfonate, preferably bromide, optionally in the form of the racemates, the enantiomers, and the hydrates thereof.

Particularly preferred according to the invention is the salt of formula 1a wherein X⁻ denotes bromide.

Of particular interest according to the invention are the enantiomers of formula 1a-en 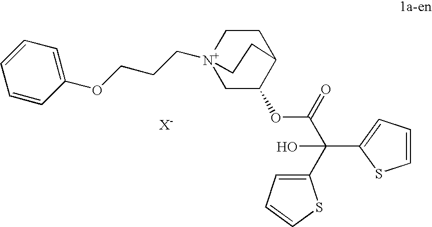
wherein X⁻ may have the meanings as mentioned hereinbefore.

In a yet another preferred embodiment according to the invention, the anticholinergic agents 1 are represented by the compounds of formula 1b 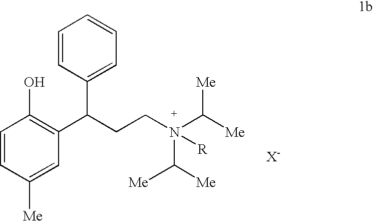
wherein R is either methyl or ethyl and wherein X⁻ may have the meanings as mentioned hereinbefore. In the alternative, the compound according to formula 1b may be present in form of its free base according to formula 1b-base

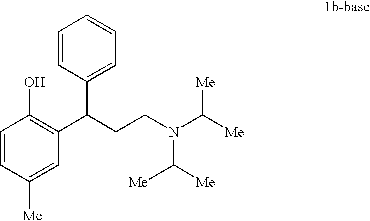

The pharmaceutical combinations according to the invention may contain the anticholinergic agent of formula 1b (or 1b-base) in form of their enantiomers, mixtures of enantiomers, or racemates. Preferably, the anticholinergic agent of formula 1b (or 1b-base) is present in form of its R-enantiomer.

Within the scope of the present invention the anticholinergic agents 1 are in a yet another preferred embodiment selected from the compounds of formula 1c 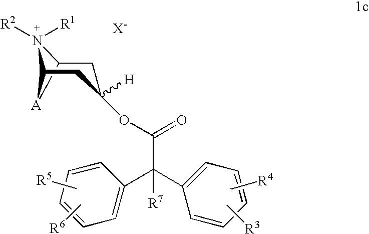
wherein:
A denotes a double-bonded group selected from among 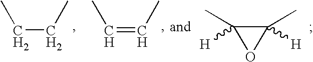
X⁻ may have the meanings as mentioned hereinbefore, preferably chloride, bromide, or methanesulfonate,
R¹ and R², which may be identical or different, denote a group selected from among methyl, ethyl, n-propyl, and isopropyl, which may optionally be substituted by hydroxy or fluorine, preferably unsubstituted methyl;
R³, R⁴, R⁵, and R⁶, which may be identical or different, denote hydrogen, methyl, ethyl, methyloxy, ethyloxy, hydroxy, fluorine, chlorine, bromine, —CN, —CF₃, or —NO₂; and
R⁷ denotes hydrogen, methyl, ethyl, methyloxy, ethyloxy, —CH₂F, —CH₂CH₂F, —OCH₂F, —OCH₂CH₂F, —CH₂OH, —CH₂CH₂OH, —CF₃, —CH₂OMe, —CH₂CH₂OMe, —CH₂OEt, —CH₂CH₂OEt, —O—COMe, —O—COEt, —O—COCF₃, —O—COCF₃, fluorine, chlorine, or bromine.

The compounds of formula 1c are known in the art (WO 02/32899, corresponding to U.S. Pat. No. 6,706,726, which is hereby incorporated by reference).

Preferred compounds of formula 1c within the combinations according to the invention are those, wherein:
X⁻ denotes bromide;
R¹ and R², which may be identical or different, denote a group selected from methyl and ethyl, preferably methyl;
R³, R⁴, R⁵, and R⁶, which may be identical or different, denote hydrogen, methyl, methyloxy, chlorine, or fluorine; and
R⁷ denotes hydrogen, methyl, or fluorine.

Of particular importance within the combinations according to the invention are compounds of general formula 1c, wherein: A denotes a double-bonded group selected from among

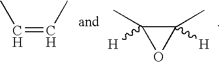

The compounds of formula 1c, may optionally be present in the form of the individual optical isomers, mixtures of the individual enantiomers or racemates thereof.

Of particular importance are those pharmaceutical compositions that contain the compound of formula 2 in combination with one of the following compounds 1c:
tropenol 2,2-diphenylpropionic acid ester methobromide,
scopine 2,2-diphenylpropionic acid ester methobromide,
scopine 2-fluoro-2,2-diphenylacetic acid ester methobromide, and
tropenol 2-fluoro-2,2-diphenylacetic acid ester methobromide.

Within the scope of the present invention, the anticholinergic agents 1 are in a yet another preferred embodiment selected from the compounds of formula 1d 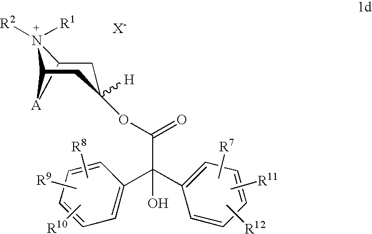
wherein:
A, X⁻, R¹, and R² may have the meanings as mentioned hereinbefore; and
R⁷, R⁸, R⁹, R¹⁰, R¹¹, and R¹², which may be identical or different, denote hydrogen, methyl, ethyl, methyloxy, ethyloxy, hydroxy, fluorine, chlorine, bromine, CN, CF₃, or NO₂, with the proviso that at least one of the groups R⁷, R⁸, R⁹, R¹⁰, R¹¹, and R¹² is not hydrogen.

The compounds of formula 1d are known in the art (WO 02/32898).

Particularly preferred within the combinations according to the invention are compounds of general formula 1d, wherein:
A denotes a double-bonded group selected from among 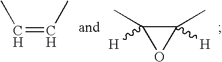
X⁻ denotes bromide;
R¹ and R², which may be identical or different, denote methyl or ethyl, preferably methyl; and
R⁷, R⁸, R⁹, R¹⁰, R¹¹, and R¹², which may be identical or different, denote hydrogen, fluorine, chlorine, or bromine, preferably fluorine with the proviso that at least one of the groups R⁷, R⁸, R⁹, R¹⁰, R¹¹, and R¹² not hydrogen.

Of particular importance are those pharmaceutical compositions that contain the compound of formula 2 in combination with one of the following compounds 1d:
tropenol 3,3′,4,4′-tetrafluorobenzilic acid ester methobromide,
scopine 3,3′,4,4′-tetrafluorobenzilic acid ester methobromide,
scopine 4,4′-difluorobenzilic acid ester methobromide,
tropenol 4,4′-difluorobenzilic acid ester methobromide,
scopine 3,3′-difluorobenzilic acid ester methobromide, and
tropenol 3,3′-difluorobenzilic acid ester methobromide.

The pharmaceutical compositions according to the invention may contain the compounds of formula 1d optionally in the form of the individual optical isomers, mixtures of the individual enantiomers or racemates thereof.

Within the scope of the present invention, the anticholinergic agents 1 are in a yet another preferred embodiment selected from the compounds of formula 1e 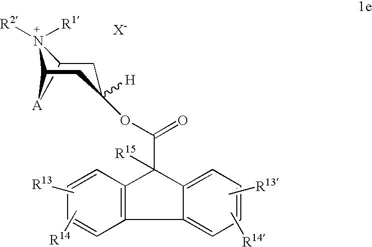
wherein:
A and X⁻ may have the meanings as mentioned hereinbefore;
R¹⁵ denotes hydrogen, hydroxy, methyl, ethyl, —CF₃, —CHF₂, or fluorine;
R^{1′} and R^{2′}, which may be identical or different, denote C₁-C₅-alkyl which may optionally be substituted by C₃-C₆-cycloalkyl, hydroxy or halogen, or R^{1′} and R^{2′} together denote a —C₃-C₅-alkylene-bridge; and
R¹³, R¹⁴, R^{13′}, and R^{14′}, which may be identical or different, denote hydrogen, —C₁-C₄-alkyl, —C₁-C₄-alkyloxy, hydroxy, —CF₃, —CHF₂, —CN, —NO₂, or halogen.

The compounds of formula 1e are not yet known in the art.

Particularly preferred within the combinations according to the invention are compounds of general formula 1e, wherein:
A denotes a double-bonded group selected from among 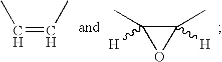
X⁻ denotes an anion selected from among chloride, bromide, and methanesulfonate, preferably bromide;
R¹⁵ denotes hydroxy, methyl, or fluorine, preferably methyl or hydroxy;
R^{1′} and R^{2′}, which may be identical or different, represent methyl or ethyl, preferably methyl; and
R¹³, R¹⁴, R^{13′}, and R^{14′}, which may be identical or different, represent hydrogen, —CF₃, —CHF₂, or fluorine, preferably hydrogen or fluorine.

Particularly preferred within the combinations according to the invention are compounds of general formula 1e, wherein
A denotes a double-bonded group selected from among 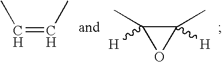
X⁻ denotes bromide;
R¹⁵ denotes hydroxy or methyl, preferably methyl;
R^{1′} and R²′, which may be identical or different, represent methyl or ethyl, preferably methyl; and
R¹³, R¹⁴, R^{13′}, and R^{14′}, which may be identical or different, represent hydrogen or fluorine.

Of particular importance are those pharmaceutical compositions that contain the compound of formula 2 in combination with one of the following compounds 1e:
tropenol 9-hydroxyfluorene-9-carboxylate methobromide;
tropenol 9-fluorofluorene-9-carboxylate methobromide;
scopine 9-hydroxyfluorene-9-carboxylate methobromide;
scopine 9-fluorofluorene-9-carboxylate methobromide;
tropenol 9-methylfluorene-9-carboxylate methobromide; and
scopine 9-methylfluorene-9-carboxylate methobromide.

The pharmaceutical compositions according to the invention may contain the compounds of formula 1e optionally in the form of the individual optical isomers, mixtures of the individual enantiomers, or racemates thereof.

Within the scope of the present invention, the anticholinergic agents 1 are in a yet another preferred embodiment selected from the compounds of formula 1f 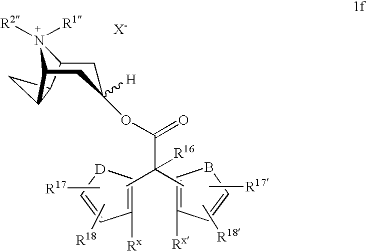
wherein X⁻ may have the meanings as mentioned hereinbefore, and wherein
D and B, which may be identical or different, preferably identical, denote —O—, —S—, —NH—, —CH₂—, —CH═CH—, or —N(C₁-C₄-alkyl)-;
R¹⁶ denotes hydrogen, hydroxy, —C₁-C₄-alkyl, —C₁-C₄-alkyloxy, —C₁-C₄-alkylene-halogen, —O—C₁-C₄-alkylene-halogen, —C₁-C₄-alkylene-OH, —CF₃, —CHF₂, —C₁-C₄-alkylene-C₁-C₄-alkyloxy, —O—COC₁-C₄-alkyl, —O—COC₁-C₄-alkylene-halogen, —C₁-C₄-alkylene-C₃-C₆-cycloalkyl, —O—COCF₃, or halogen;
R^{1″} and R^{2″} which may be identical or different, denote —C₁-C₅-alkyl, which may optionally be substituted by —C₃-C₆-cycloalkyl, hydroxyl, or halogen, or R^{1″} and R^{2″} together denote a —C₃-C₅-alkylene bridge;
R¹⁷, R¹⁸, R^{17′}, and R^{18′}, which may be identical or different, denote hydrogen, C₁-C₄-alkyl, C₁-C₄-alkyloxy, hydroxy, —CF₃, —CHF₂, —CN, —NO₂, or halogen; and
R^{x} and R^{x′}, which may be identical or different, denote hydrogen, C₁-C₄-alkyl, C₁-C₄-alkyloxy, hydroxy, —CF₃, —CHF₂, —CN, —NO₂, or halogen, or R^{x} and R^{x′} together denote a single bond or a bridging group selected from among the bridges —O—, —S—, —NH—, —CH₂—, —CH₂—CH₂—, —N(C₁-C₄-alkyl)-, —CH(C₁-C₄-alkyl)-, and —C(C₁-C₄-alkyl)₂.

The compounds of formula 1f are not yet known in the art.

Particularly preferred within the combinations according to the invention are compounds of general formula 1f wherein:
X⁻ denotes chloride, bromide, or methanesulfonate, preferably bromide;
D and B, which may be identical or different, preferably identical, denote —O—, —S—, —NH—, or —CH═CH—;
R¹⁶ denotes hydrogen, hydroxy, —C₁-C₄-alkyl, —C₁-C₄-alkyloxy, —CF₃, —CHF₂, fluorine, chlorine, or bromine;
R^{1″} and R^{2″}, which may be identical or different, denote C₁-C₄-alkyl, which may optionally be substituted by hydroxy, fluorine, chlorine, or bromine, or R^{1″} and R^{2″} together denote a —C₃-C₄-alkylene-bridge;
R¹⁷, R¹⁸, R^{17′}, and R^{18 ′}, which may be identical or different, denote hydrogen, C₁-C₄-alkyl, C₁-C₄-alkyloxy, hydroxy, —CF₃, —CHF₂, —CN, —NO₂, fluorine, chlorine, or bromine; and
R^{x} and R^{x′} which may be identical or different, denote hydrogen, C₁-C₄-alkyl, C₁-C₄-alkyloxy, hydroxy, —CF₃, —CHF₂, —CN, —NO₂, fluorine, chlorine, or bromine, or R^{x} and R^{x′} together denote a single bond or a bridging group selected from among the bridges —O—, —S—, —NH—, and —CH₂—.

Particularly preferred within the combinations according to the invention are compounds of general formula 1f, wherein:
X⁻ denotes chloride, bromide, or methanesulfonate, preferably bromide;
D and B, which may be identical or different, preferably identical, denote —S— or —CH═CH—;
R¹⁶ denotes hydrogen, hydroxyl, or methyl;
R^{1″} and R^{2″}, which may be identical or different, denote methyl or ethyl;
R¹⁷, R¹⁸, R^{17′}, and R^{18′}, which may be identical or different, denote hydrogen, —CF₃ or fluorine, preferably hydrogen; and
R^{x} and R^{x′}, which may be identical or different, denote hydrogen, —CF₃, or fluorine, preferably hydrogen, or R^{x} and R^{x′} together denote a single bond or the bridging group —O—.

Particularly preferred within the combinations according to the invention are compounds of general formula 1f, wherein:
X⁻ denotes bromide;
D and B denote —CH═CH—;
R¹⁶ denotes hydrogen, hydroxy or methyl;
R^{1″} and R^{2″} denote methyl;
R¹⁷, R¹⁸ , R^{17′}, and R^{18′}, which may be identical or different, denote hydrogen or fluorine, preferably hydrogen; and
R^{x} and R^{x′}, which may be identical or different, denote hydrogen or fluorine, preferably hydrogen or R^{x} and R^{x′} together denote a single bond or the bridging group —O—.

Of particular importance are those pharmaceutical compositions that contain the compound of formula 2 in combination with one of the following compounds 1f:
cyclopropyltropine benzilate methobromide
cyclopropyltropine 2,2-diphenylpropionate methobromide;
cyclopropyltropine 9-hydroxyxanthene-9-carboxylate methobromide; cyclopropyltropine 9-methylfluorene-9-carboxylate methobromide;
cyclopropyltropine 9-methylxanthene-9-carboxylate methobromide
cyclopropyltropine 9-hydroxyfluorene-9-carboxylate methobromide; and
cyclopropyltropine methyl 4,4′-difluorobenzilate methobromide.

The pharmaceutical compositions according to the invention may contain the compounds of formula 1f optionally in the form of the individual optical isomers, mixtures of the individual enantiomers, or racemates thereof.

Within the scope of the present invention the anticholinergic agents 1 are in a yet another preferred embodiment selected from the compounds of formula 1g 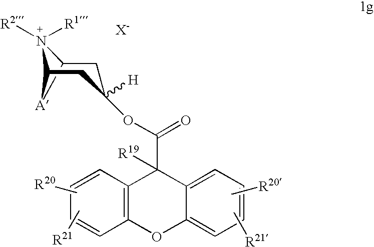
wherein X⁻ may have the meanings as mentioned hereinbefore, and wherein
A′ denotes a double-bonded group selected from among 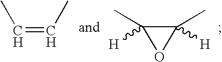
R¹⁹ denotes hydroxy, methyl, hydroxymethyl, ethyl, —CF₃, —CHF₂, or fluorine;
R^{1′″} and R^{2′″}, which may be identical or different, denote C₁-C₅-alkyl which may optionally be substituted by C₃-C₆-cycloalkyl, hydroxyl, or halogen, or R^{1′″} and R^{2′″} together denote a —C₃-C₅-alkylene-bridge; and
R²⁰, R²¹, R^{20′}, and R^{21′}, which may be identical or different, denote hydrogen, —C₁-C₄-alkyl, —C₁-C₄-alkyloxy, hydroxy, —CF₃, —CHF₂, —CN, —NO₂, or halogen.

The compounds of formula 1g are not yet known in the art.

Particularly preferred within the combinations according to the invention are compounds of general formula 1g wherein:
A′ denotes a double-bonded group selected from among 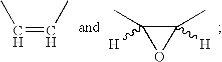
X⁻ denotes chloride, bromide, or methanesulfonate, preferably bromide;
R¹⁹ denotes hydroxy or methyl;
R^{1′″} and R^{2′″}, which may be identical or different, represent methyl or ethyl, preferably methyl; and
R²⁰, R²¹, R^{20′}, and R^{21′}, which may be identical or different, represent hydrogen, —CF₃, —CHF₂, or fluorine, preferably hydrogen or fluorine.

Particularly preferred within the combinations according to the invention are compounds of general formula 1g wherein

A′ denotes a double-bonded group selected from among 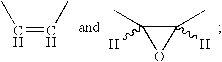
X⁻ denotes bromide;
R¹⁹ denotes hydroxy or methyl, preferably methyl;
R^{1′″} and R^{2′″}, which may be identical or different, represent methyl or ethyl, preferably methyl; and
R³, R⁴, R^{3′}, and R^{4′}, which may be identical or different, represent hydrogen or fluorine.

Of particular importance are those pharmaceutical compositions that contain the compound of formula 2 in combination with one of the following compounds 1g:
tropenol 9-hydroxyxanthene-9-carboxylate methobromide;
scopine 9-hydroxyxanthene-9-carboxylate methobromide;
tropenol 9-methylxanthene-9-carboxylate methobromide;
scopine 9-methylxanthene-9-carboxylate methobromide;
tropenol 9-ethylxanthene-9-carboxylate methobromide;
tropenol 9-difluoromethylxanthene-9-carboxylate methobromide; and
scopine 9-hydroxymethylxanthene-9-carboxylate methobromide.

The pharmaceutical compositions according to the invention may contain the compounds of formula 1g optionally in the form of the individual optical isomers, mixtures of the individual enantiomers or racemates thereof.

The alkyl groups used, unless otherwise stated, are branched and unbranched alkyl groups having 1 to 5 carbon atoms. Examples include: methyl, ethyl, propyl, or butyl. The groups methyl, ethyl, propyl, or butyl may optionally also be referred to by the abbreviations Me, Et, Prop, or Bu. Unless otherwise stated, the definitions propyl and butyl also include all possible isomeric forms of the groups in question. Thus, for example, propyl includes n-propyl and isopropyl, butyl includes isobutyl, sec-butyl, and tert-butyl, etc.

The cycloalkyl groups used, unless otherwise stated, are alicyclic groups with 3 to 6 carbon atoms. These are the cyclopropyl, cyclobutyl, cyclopentyl, and cyclohexyl groups. According to the invention cyclopropyl is of particular importance within the scope of the present invention.

The alkylene groups used, unless otherwise stated, are branched and unbranched double-bonded alkyl bridges with 1 to 5 carbon atoms. Examples include: methylene, ethylene, propylene, or butylene.

The alkylene-halogen groups used, unless otherwise stated, are branched and unbranched double-bonded alkyl bridges with 1 to 4 carbon atoms which may be mono-, di-, or trisubstituted, preferably disubstituted, by a halogen. Accordingly, unless otherwise stated, the term alkylene-OH groups denotes branched and unbranched double-bonded alkyl bridges with 1 to 4 carbon atoms which may be mono-, di-, or trisubstituted, preferably monosubstituted, by a hydroxy.

The alkyloxy groups used, unless otherwise stated, are branched and unbranched alkyl groups with 1 to 5 carbon atoms which are linked via an oxygen atom. The following may be mentioned, for example: methyloxy, ethyloxy, propyloxy, or butyloxy. The groups methyloxy, ethyloxy, propyloxy, or butyloxy may optionally also be referred to by the abbreviations MeO, EtO, PropO, or BuO. Unless otherwise stated, the definitions propyloxy and butyloxy also include all possible isomeric forms of the groups in question. Thus, for example, propyloxy includes n-propyloxy and isopropyloxy, butyloxy includes isobutyloxy, sec-butyloxy, and tert-butyloxy, etc. The word alkoxy may also possibly be used within the scope of the present invention instead of the word alkyloxy. The groups methyloxy, ethyloxy, propyloxy, or butyloxy may optionally also be referred to as methoxy, ethoxy, propoxy, or butoxy.

The alkylene-alkyloxy groups used, unless otherwise stated, are branched and unbranched double-bonded alkyl bridges with 1 to 5 carbon atoms which may be mono-, di-, or trisubstituted, preferably monosubstituted, by an alkyloxy group.

The —O—CO-alkyl groups used, unless otherwise stated, are branched and unbranched alkyl groups with 1 to 4 carbon atoms which are bonded via an ester group. The alkyl groups are bonded directly to the carbonyl carbon of the ester group. The term —O—CO-alkyl-halogen group should be understood analogously. The group —O—CO—CF₃ denotes trifluoroacetate.

Within the scope of the present invention halogen denotes fluorine, chlorine, bromine, or iodine. Unless otherwise stated, fluorine and bromine are the preferred halogens. The group CO denotes a carbonyl group.

Surprisingly, an unexpectedly beneficial therapeutic effect can be observed in the treatment of inflammatory and/or obstructive diseases of the respiratory tract if an anticholinergic 1 is used with the betamimetic of formula 2.

The beneficial therapeutic effect mentioned above may be observed both when the two active substances are administered simultaneously in a single active substance formulation and when they are administered successively in separate formulations. According to the invention, it is preferable to administer the two active substance ingredients simultaneously in a single formulation.

Within the scope of the present invention, any reference to the compounds 1′ is to be regarded as a reference to the pharmacologically active cations contained in the salts 1. These are the cations tiotropium, oxitropium, flutropium, ipratropium, glycopyrronium, trospium, or the cations of the following formulae

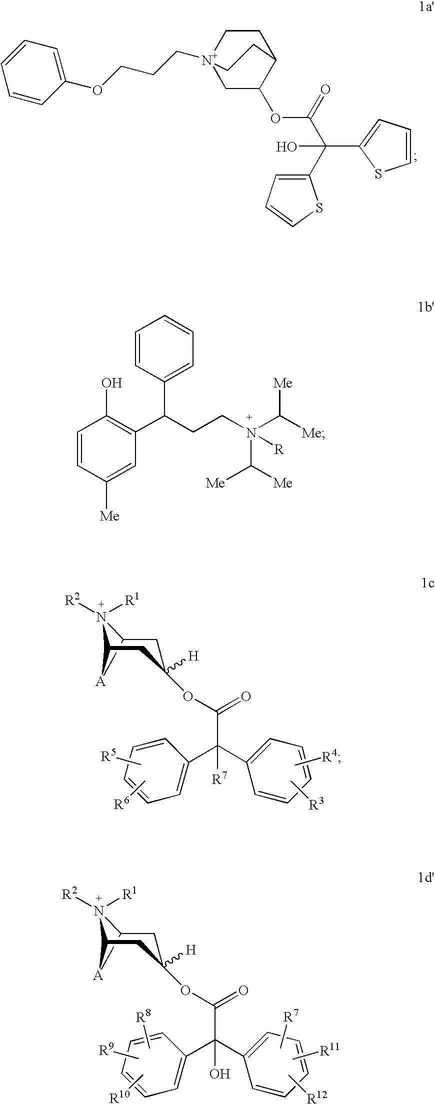

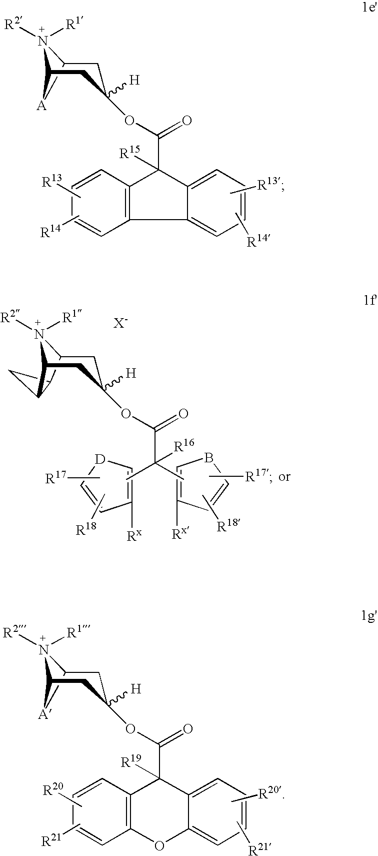

In the pharmaceutical combinations mentioned above, the active substances may be combined in a single preparation or contained in two separate formulations.

Pharmaceutical compositions which contain the active substances 1 and 2 in a single preparation are preferred according to the invention.

In one aspect, the present invention relates to the abovementioned pharmaceutical compositions which contain, in addition to therapeutically effective quantities of 1 and 2, a pharmaceutically acceptable carrier. In another aspect the present invention relates to the abovementioned pharmaceutical compositions which do not contain any pharmaceutically acceptable carrier in addition to therapeutically effective quantities of 1 and 2.

The present invention also relates to the use of therapeutically effective quantities of the salts 1 for preparing a pharmaceutical composition also containing 2 for treating inflammatory or obstructive diseases of the respiratory tract. Preferably, the present invention relates to the abovementioned use for preparing a pharmaceutical composition for treating asthma or COPD.

Within the scope of the present invention the compounds 1 and 2 may be administered simultaneously or successively, while it is preferable according to the invention to administer compounds 1 and 2 simultaneously.

The present invention further relates to the use of therapeutically effect amounts of 1 and 2 for treating inflammatory or obstructive respiratory complaints, particularly asthma or COPD.

The proportions in which the active substances 1 and 2 may be used in the active substance combinations according to the invention are variable. Active substances 1 and 2 may possibly be present in the form of their solvates or hydrates. Depending on the choice of the compounds 1 and 2, the weight ratios which may be used within the scope of the present invention vary on the basis of the different molecular weights of the various compounds and their different potencies. In general, the combinations according to the invention may contain the components 1 and 2 generally in weight ratios in the range from 1:400 to 150:1, preferably in a weight ratio in the range from 1:350 to 100:1.

The pharmaceutical compositions according to the invention containing the combinations of 1 and 2 are normally used so that 1 and 2 (values based on free base) are administered together in doses of 0.01 μg to 10000 μg, preferably 0.1 μg to 5000 μg, particularly preferably from 0.5 μg to 1000 μg per single dose.

In case the composition according to the invention contains a tiotropium salt as the anticholinergic component 1, the combination of active substances according to the invention may contain tiotropium cation 1′ and the compound of formula 2 (based on free base) in the range from 1:300 to 50:1, preferably from 1:200 to 30:1, particularly preferably from 1:150 to 20:1, more preferably from 1:50 to 15:1. For example, without restricting the scope of the invention, preferred combinations of 1 and 2 according to the invention may contain tiotropium 1′ and 2 (values based on free base) in the following weight ratios: 1:35, 1:34, 1:33, 1:32, 1:31, 1:30, 1:29, 1:29, 1:27, 1:26, 1:25, 1:24, 1:23, 1:22, 1:21, 1:20, 1:19, 1:18, 1:17, 1:16, 1:15, 1:14, 1:13, 1:12, 1:11, 1:10, 1:9, 1:8, 1:7, 1:6, 1:5, 1:4, 1:3, 1:2, 1:1, 2:1, 3:1, 4:1, 5:1, 6:1, 7:1, 8:1, 9:1, 10:1, 11:1, 12:1, 13:1, 14:1, and 15:1.

The pharmaceutical compositions according to the invention containing the combinations of tiotropium as ingredient 1 and 2 are preferably administered so that 1′ (tiotropium cation) and 2 (values based on free base) are present together in dosages of 5 μg to 500 μg, preferably, according to the invention, from 10 μg to 200 μg per single dose.

For example, combinations of 1 and 2 according to the invention contain an amount of tiotropium 1′ and compound 2 (values based on free base) such that the total dosage per single dose is 10 μg, 15 μg, 20 μg, 25 μg, 30 μg, 35 μg, 45 μg, 50 μg, 55 μg, 60 μg, 65 μg, 70 μg, 75 μg, 80 μg, 85 μg, 90 μg, 95 μg, 100 μg, 105 μg, 110 μg, 115 μg, 120 μg, 125 μg, 130 μg, 135 μg, 140 μg, 145 μg, 150 μg, 155 μg, 160 μg, 165 μg, 170 μg, 175 μg, 180 μg, 185 μg, 190 μg, 195 μg, 200 μg, or similar. It is clear to anyone skilled in the art that the suggested dosages per single dose specified above are not to be regarded as being limited to the numerical values actually stated. Fluctuations of about ±2.5 μg, particularly in the decimal range, are also included, as will be apparent to one of skill in the art. In these dosage ranges, the active substances 1′ and 2 may be present in the weight ratios given above.

For example, without restricting the scope of the invention, the combinations of 1 and 2 according to the invention may contain an amount of tiotropium 1′ and compound 2 (values based on free base) such that 5 μg of 1′ and 5 μg of 2, 5 μg of 1′ and 10 μg of 2, 5 μg of 1′ and 15 μg of 2, 5 μg of 1′ and 25 μg of 2, 5 μg of 1′ and 50 μg of 2, 5 μg of 1′ and 100 μg of 2, 10 μg of 1′ and 5 μg of 2, 10 μg of 1′ and 10 μg of 2, 10 μg of 1′ and 15 μg of 2, 10 μg of 1′ and 25 μg of 2, 10 μg of 1′ and 50 μg of 2, 10 μg of 1′ and 100 μg of 2, 18 μg of 1′ and 5 μg of 2, 18 μg of 1′ and 10 μg of 2, 18μg of 1′ and 15 μg of 2, 18 μg of 1′ and 25 μg of 2, 18 μg of 1′ and 50 μg of 2, 18 μg of 1′ and 100 μg of 2, 36 μg of 1′0 and 5 μg of 2, 36 μg of 140 and 10 μg of 2, 36 μg of 1′ and 15 μg of 2, 36 μg of 1′ and 25 μg of 2, 36 μg of 1′ and 50 μg of 2, 36 μg of 1′ and 100 μg of 2, 40 μg of 1′ and 5 μg of 2, 40 μg of 1′ and 10 μg of 2, 40 μg of 1′ and 15 μg of 2, 40 μg of 1′ and 25 μg of 2, 40 μg of 1′ and 50 μg of 2, or 40 μg of 1′ and 100 μg of 2 are administered per single dose.

From the aforementioned examples for suitable doses of the tiotropium containing combinations according to the invention, the corresponding amounts of the salts 1 and of the acid addition salts of 2 are readily calculable.

In case the composition according to the invention contains a salt of formula 1a as the anticholinergic component 1, the combination of active substances according to the invention may contain cation 1a′ and the compound of formula 2 (based on free base), for example,inthefollowingratiosbyweight: 1:15, 1:14, 1:13, 1:12, 1:11, 1:10, 1:9, 1:8, 1:7, 1:6, 1:5, 1:4, 1:3, 1:2, 1:1, 2:1, 3:1, 4:1, 5:1, 6:1, 7:1, 8:1, 9:1, 10:1, 11:1, 12:1, 13:1, 14:1, 15:1, 16:1, 17:1, 18:1, 19:1, 20:1, 21:1, 22:1, 23:1, 24:1, 25:1, 26:1, 27:1, 28:1, 29:1, 30:1, 31:1, 32:1, 33:1, 34:1, and 35:1.

The pharmaceutical compositions according to the invention containing the combinations of 1a and 2 are preferably administered so that 1a′ and 2 (values based on free base) are present together in dosages of 10 μg to 2000 μg, preferably from 15 μg to 1000 μg, even more preferably from 20 μg to 900 μg per single dose.

For example, combinations of 1a and 2 according to the invention contain an amount of the 1a′ and 2 (based on free base) such that the total dosage per single dose is about 15 μg, 20 μg, 25 μg, 30 μg, 35 μg, 45 μg, 50 μg, 55 μg, 60 μg, 65 μg, 70 μg, 75 μg, 80 μg, 85 μg, 90 μg, 95 μg, 100 μg, 105 μg, 110 μg, 115 μg, 120 μg, 125 μg, 130 μg, 135 μg, 140 μg, 145 μg, 150 μg, 155 μg, 160 μg, 165 μg, 170 μg, 175 μg, 180 μg, 185 μg, 190 μg, 195 μg, 200 μg, 205 μg, 210 μg, 215 μg, 220 μg, 225 μg, 230 μg, 235 μg, 240 μg, 245 μg, 250 μg, 255 μg, 260 μg, 265 μg, 270 μg, 275 μg, 280 μg, 285 μg, 290 μg, 295 μg, 300 μg, 305 μg, 310 μg, 315 μg, 320 μg, 325 μg, 330 μg, 335 μg, 340 μg, 345 μg, 350 μg, 355 μg, 360 μg, 365 μg, 370 μg, 375 μg, 380 μg, 385 μg, 390 μg, 395 μg, 400 μg, 405 μg, 410 μg, 415 μg, 420 μg, 425 μg, 430 μg, 435 μg, 440 μg, 445 μg, 450 μg, 455 μg, 460 μg, 465 μg, 470 μg, 475 μg, 480 μg, 485 μg, 490 μg, 495 μg, 500 μg, 505 μg, 510 μg, 515 μg, 520 μg, 525 μg, 530 μg, 535 μg, 540 μg, 545 μg, 550 μg, 555 μg, 560 μg, 565 μg, 570 μg, 575 μg, 580 μg, 585 μg, 590 μg, 595 μg, 600 μg, 605 μg, 610 μg, 615 μg, 620 μg, 625 μg, 630 μg, 635 μg, 640 μg, 645 μg, 650 μg, 655 μg, 660 μg, 665 μg, 670 μg, 675 μg, 680 μg, 685 μg, 690 μg, 695 μg, 700 μg, 605 μg, 610 μg, 615 μg, 620 μg, 625 μg, 630 μg, 635 μg, 640 μg, 645 μg, 650 μg, 655 μg, 660 μg, 665 μg, 670 μg, 675 μg, 680 μg, 685 μg, 690 μg, 695 μg, 700 μg, 705 μg, 710 μg, 715 μg, 720 μg, 725 μg, 730 μg, 735 μg, 740 μg, 745 μg, 750 μg, 755 μg, 760 μg, 765 μg, 770 μg, 775 μg, 780 μg, 785 μg, 790 μg, 795 μg, 800 μg, 805 μg, 810 μg, 815 μg, 820 μg, 825 μg, 830 μg, 835 μg, 840 μg, 845 μg, 850 μg, 855 μg, 860 μg, 865 μg, 870 μg, 875 μg, 880 μg, 885 μg, 890 μg, 895 μg, 900 μg, or similar. It is clear to anyone skilled in the art that the suggested dosages per single dose specified above are not to be regarded as being limited to the numerical values actually stated. Fluctuations of about ±2.5 μg, particularly in the decimal range, are also included, as will be apparent to one of skill in the art. In these dosage ranges, the active substances 1a′ and 2 may be present in the weight ratios given above.

For example, without restricting the scope of the invention thereto, the pharmaceutical compositions according to the invention may contain for instance the following quantities for each single dose: 20 μg of 1a′ and 5 μg of 2, 20 μg of 1a′ and 10 μg of 2, 20 μg of 1′ and 15 μg of 2, 20 μg of 1a′ and 25 μg of 2, 20 μg of 1a′ and 50 μg of 2, 20 μg of 1a′ and 100 μg of 2, 40 μg of 1a′ and 5 μg of 2, 40 μg of 1a′ and 10 μg of 2, 40μg of 1a′ and 15 μg of 2, 40 μg of 1a′ and 25 μg of 2, 40 μg of 1a′ and 50 μg of 2, 40 μg of 1a′ and 100 μg of 2, 60 μg of 1a′ and 5 μg of 2, 60 μg of 1a′ and 10 μg of 2, 60 μg of 1a′ and 15 μg of 2, 60 μg of 1a′ and 25 μg of 2, 60 μg of 1a′ and 50 μg of 2, 60 μg of 1a′ and 100 μg of 2, 100 μg of 1a′ and 5 μg of 2, 100 μg of 1a′ and 10 μg of 2, 100 μg of 1a′ and 15 μg of 2, 100 μg of 1a′ and 25 μg of 2, 100 μg of 1a′ and 50 μg of 2, 100 μg of 1a′ and 100 μg of 2, 200 μg of 1a′ and 5 μg of 2, 200 μg of 1a′ and 10 μg of 2, 200 μg of 1a′ and 15 μg of 2, 200 μg of 1a′ and 25 μg of 2, 200 μg of 1a′ and 50 μg of 2, 200 μg of 1a′ and 100 μg of 2, 300 μg of 1a′ and 5 μg of 2, 300 μg of 1a′ and 10 μg of 2, 300 μg of 1a′ and 15 μg of 2, 300 μg of 1a′ and 25 μg of 2, 300 μg of 1a′ and 50 μg of 2, 300 μg of 1a′ and 100 μg of 2, 400 μg of 1a′ and 5 μg of 2, 400 μg of 1a′ and 10 μg of 2, 400 μg of 1a′ and 15 μg of 2, 400 μg of 1a′ and 25 μg of 2, 400 μg of 1a′ and 50 μg of 2, 400 μg of 1a′ and 100 μg of 2, 500 μg of 1a′ and 5 μg of 2, 500 μg of 1a′ and 10 μg of 2, 500 μg of 1a′ and 15 μg of 2, 500 μg of 1a′ and 25 μg of 2, 500 μg of 1a′ and 50 μg of 2, 500 μg of 1a′ and 100 μg of 2, 600 μg of 1a′ and 5 μg of 2, 600 μg of 1a′ and 10 μg of 2, 600 μg of 1a′ and 15 μg of 2, 600 μg of 1a′ and 25 μg of 2, 600 μg of 1a′ and 50 μg of 2, 600 μg of 1a′ and 100 μg of 2, 700 μg of 1a′ and 5 μg of 2, 700 μg of 1a′ and 10 μg of 2, 700 μg of 1a′ and 15 μg of 2, 700 μg of 1a′ and 25 μg of 2, 700 μg of 1a′ and 50 μg of 2, 700 μg of 1a′ and 100 μg of 2, 800 μg of 1a′ and 5 μg of 2, 800 μg of 1a′ and 10 μg of 2, 800 μg of 1a′ and 15 μg of 2, 800 μg of 1a′ and 25 μg of 2, 800 μg of 1a′ and 50 μg of 2, 800 μg of 1a′ and 100 μg of 2, 900 μg of 1a′ and 5 μg of 2, 900 μg of 1a′ and 10 μg of 2, 900 μg of 1a′ and 15 μg of 2 900 μg of 1a′ and 25 μg of 2, 900 μg of 1a′ and 50 μg of 2, 900 μg of 1a′ and 100 μg of 2, 1000 μg of 1a′ and 5 μg of 2, 1000 μg of 1a′ and 10 μg of 2, 1000 μg of 1a′ and 15 μg of 2, 1000 μg of 1a′ and 25 μg of 2, 1000 μg of 1a′ and 50 μg of 2, and 1000 μg of 1a′ and 100 μg of 2.

From the aforementioned examples for suitable doses of the 1a′ containing combinations according to the invention, the corresponding amounts of the salts 1a and of the acid addition salts of 2 are readily calculable.

In case the composition according to the invention contains a salt of formula 1c as the anticholinergic component 1, the combination of active substances according to the invention may contain cation 1c′ and the compound of formula 2 (based on free base) for example in the following ratios by weight: 1:15, 1:14, 1:13, 1:12, 1:11, 1:10, 1:9, 1:8, 1:7, 1:6, 1:5, 1:4, 1:3, 1:2, 1:1, 2:1, 3:1, 4:1, 5:1, 6:1, 7:1, 8:1, 9:1, 10:1, 11:1, 12:1, 13:1, 14:1, 15:1, 16:1, 17:1, 18:1, 19:1, 20:1, 21:1, 22:1, 23:1, 24:1, 25:1, 26:1, 27:1, 28:1, 29:1, 30:1, 31:1, 32:1, 33:1, 34:1, and 35:1.

The pharmaceutical compositions according to the invention containing the combinations of 1c and 2 are preferably administered so that the cation 1c′ and 2 (values based on free base) are present together in dosages of 10 μg to 2000 μg, more preferably from 15 μg to 1000 μg, even more preferably from 20 μg to 800 μg, and preferably according to the invention from 30 μg to 750 μg, preferably from 40 μg to 700 μg per single dose.

For example, combinations of 1c and 2 according to the invention contain an amount of 1c′ and 2 (values based on free base) such that the total dosage per single dose is about 15 μg, 20 μg, 25 μg, 30 μg, 35 μg, 45 μg, 50 μg, 55 μg, 60 μg, 65 μg, 70 μg, 75 μg, 80 μg, 85 μg, 90 μg, 95 μg, 100 μg, 105 μg, 110 μg, 115 μg, 120 μg, 125 μg, 130 μg, 135 μg, 140 μg, 145 μg, 150 μg, 155 μg, 160 μg, 165 μg, 170 μg, 175 μg, 180 μg, 185 μg, 190 μg, 195 μg, 200 μg, 205 μg, 210 μg, 215 μg, 220 μg, 225 μg, 230 μg, 235 μg, 240 μg, 245 μg, 250 μg, 255 μg, 260 μg, 265 μg, 270 μg, 275 μg, 280 μg, 285 μg, 290 μg, 295 μg, 300 μg, 305 μg, 310 μg, 315 μg, 320 μg, 325 μg, 330 μg, 335 μg, 340 μg, 345 μg, 350 μg, 355 μg, 360 μg, 365 μg, 370 μg, 375 μg, 380 μg, 385 μg, 390 μg, 395 μg, 400 μg, 405 μg, 410 μg, 415 μg, 420 μg, 425 μg, 430 μg, 435 μg, 440 μg, 445 μg, 450 μg, 455 μg, 460 μg, 465 μg, 470 μg, 475 μg, 480 μg, 485 μg, 490 μg, 495 μg, 500 μg, 505 μg, 510 μg, 515 μg, 520 μg, 525 μg, 530 μg, 535 μg, 540 μg, 545 μg, 550 μg, 555 μg, 560 μg, 565 μg, 570 μg, 575 μg, 580 μg, 585 μg, 590 μg, 595 μg, 600 μg, 605 μg, 610 μg, 615 μg, 620 μg, 625 μg, 630 μg, 635 μg, 640 μg, 645 μg, 650 μg, 655 μg, 660 μg, 665 μg, 670 μg, 675 μg, 680 μg, 685 μg, 690 μg, 695 μg, 700 μg, 605 μg, 610 μg, 615 μg, 620 μg, 625 μg, 630 μg, 635 μg, 640 μg, 645 μg, 650 μg, 655 μg, 660 μg, 665 μg, 670 μg, 675 μg, 680 μg, 685 μg, 690 μg, 695 μg, 700 μg, or similar. It is clear to anyone skilled in the art that the suggested dosages per single dose specified above are not to be regarded as being limited to the numerical values actually stated. Fluctuations of about ±2.5 μg, particularly in the decimal range, are also included, as will be apparent to one of skill in the art. In these dosage ranges, the active substances 1c′ and 2 may be present in the weight ratios given above.

For example, without restricting the scope of the invention thereto, the combinations of 1c and 2 according to the invention may contain a quantity of cation 1c′ and 2 (values based on free base) such that, for each single dose, 8.3 μg of 1c′ and 5 μg of 2, 8.3 μg of 1c′ and 10 μg of 2, 8.3 μg of 1c′ and 15 μg of 2, 8.3 μg of 1c′ and 25 μg of 2, 8.3 μg of 1c′ and 50 μg of 2, 8.3 μg of 1c′ and 100 μg of 2, 16.5 μg of 1c′ and 5 μg of 2, 16.5 μg of 1c′ and 10 μg of 2, 16.5 μg of 1c′ and 15 μg of 2, 16.5 μg of 1c′ and 25 μg of 2, 16.5 μg of 1c′ and 50 μg of 2, 16.5 μg of 1c′ and 100 μg of 2, 33.0 μg of 1c′ and 5 μg of 2, 33.0 μg of 1c′ and 10 μg of 2, 33.0 μg of 1c′ and 15 μg of 2, 33.0 μg of 1c′ and 25 μg of 2, 33.0 μg of 1c′ and 50 μg of 2, 33.0 μg of 1c′ and 100 μg of 2, 49.5 μg of 1c′ and 5 μg of 2, 49.5 μg of 1c′ and 10 μg of 2, 49.5 μg of 1c′ and 15 μg of 2, 49.5 μg of 1c′ and 25 μg of 2, 49.5 μg of 1c′ and 50 μg of 2, 49.5 μg of 1c′ and 100 μg of 2, 82.6 μg of 1c′ and 5 μg of 2, 82.6 μg of 1c′ and 10 μg of 2, 82.6 μg of 1c′ and 15 μg of 2, 82.6 μg of 1c′ and 25 μg of 2, 82.6 μg of 1c′ and 50 μg of 2, 82.6 μg of 1c′ and 100 μg of 2, 165.1 μg of 1c′ and 5μg of 2, 165.1 μg of 1c′ and 10 μg of 2, 165.1 μg of 1c′ and 15 μg of 2, 165.1 μg of 1c′ and 25μg of 2, 165.1 μg of 1c′ and 50 μg of 2, 165.1 μg of 1c′ and 100 μg of 2, 206.4 μg of 1c′ and 5 μg of 2, 206.4 μg of 1c′ and 10 μg of 2, 206.4 μg of 1c′ and 15 μg of 2, 206.4 μg of 1c′ and 25 μg of 2, 206.4 μg of 1c′ and 50 μg of 2, 206.4 μg of 1c′ and 100 μg of 2, 412.8 μg of 1c′ and 5 μg of 2, 412.8 μg of 1c′ and 10 μg of 2, 412.8 μg of 1c′ and 15 μg of 2, 412.8 μg of ic′ and 25 μg of 2, 412.8 μg of 1c′ and 50 μg of 2, and 412.8 μg of 1c′ and 100 μg of 2 are present, for example.

From the aforementioned examples for suitable doses of the 1c′ containing combinations according to the invention, the corresponding amounts of the salts 1c and of the acid addition salts of 2 are readily calculable.

For compositions according to the invention that contain as the anticholinergic a compound of formula 1d the weight ratios and amounts of 1d and 2 are in the range of those suggested hereinbefore for combinations containing 1c and 2.

In case the composition according to the invention contains a salt of formula 1e as the anticholinergic component 1, the combination of active substances according to the invention may contain cation 1e′ and the compound of formula 2 (based on free base) for example in the following ratios by weight: 1:15, 1:14, 1:13, 1:12, 1:11, 1:10, 1:9, 1:8, 1:7, 1:6, 1:5, 1:4, 1:3, 1:2, 1:1, 2:1, 3:1, 4:1, 5:1, 6:1, 7:1, 8:1, 9:1, 10:1, 11:1, 12:1, 13:1, 14:1, 15:1, 16:1, 17:1, 18:1, 19:1, 20:1, 21:1, 22:1, 23:1, 24:1, 25:1, 26:1, 27:1, 28:1, 29:1, 30:1, 31:1, 32:1, 33:1, 34:1, and 35:1.

The pharmaceutical compositions according to the invention containing the combinations of 1e and 2 are preferably administered so that the cation 1e′ and 2 (values based on free base) are present together in dosages of 5 μg to 2000 μg, more preferably from 15 μg to 1000 μg, even more preferably from 20 μg to 800 μg, and preferably according to the invention from 30 μg to 750 μg, preferably from 40 μg to 700 μg per single dose. For example, combinations of 1e and 2 according to the invention contain an amount of 1e′ and 2 (values based on free base) such that the total dosage per single dose is about 15 μg, 20 μg, 25 μg, 30 μg, 35 μg, 45 μg, 50 μg, 55 μg, 60 μg, 65 μg, 70 μg, 75 μg, 80 μg, 85 μg, 90 μg, 95 μg, 100 μg, 105 μg, 110 μg, 115 μg, 120 μg, 125 μg, 130 μg, 135 μg, 140 μg, 145 μg, 150 μg, 155 μg, 160 μg, 165 μg, 170 μg, 175 μg, 180 μg, 185 μg, 190 μg, 195 μg, 200 μg, 205 μg, 210 μg, 215 μg, 220 μg, 225 μg, 230 μg, 235 μg, 240 μg, 245 μg, 250 μg, 255 μg, 260 μg, 265 μg, 270 μg, 275 μg, 280 μg, 285 μg, 290 μg, 295 μg, 300 μg, 305 μg, 310 μg, 315 μg, 320 μg, 325 μg, 330 μg, 335 μg, 340 μg, 345 μg, 350 μg, 355 μg, 360 μg, 365 μg, 370 μg, 375 μg, 380 μg, 385 μg, 390 μg, 395 μg, 400 μg, 405 μg, 410 μg, 415 μg, 420 μg, 425 μg, 430 μg, 435 μg, 440 μg, 445 μg, 450 μg, 455 μg, 460 μg, 465 μg, 470 μg, 475 μg, 480 μg, 485 μg, 490 μg, 495 μg, 500 μg, 505 μg, 510 μg, 515 μg, 520 μg, 525 μg, 530 μg, 535 μg, 540 μg, 545 μg, 550 μg, 555 μg, 560 μg, 565 μg, 570 μg, 575 μg, 580 μg, 585 μg, 590 μg, 595 μg, 600 μg, 605 μg, 610 μg, 615 μg, 620 μg, 625 μg, 630 μg, 635 μg, 640 μg, 645 μg, 650 μg, 655 μg, 660 μg, 665 μg, 670 μg, 675 μg, 680 μg, 685 μg, 690 μg, 695 μg, 700 μg, 605 μg, 610 μg, 615 μg, 620 μg, 625 μg, 630 μg, 635 μg, 640 μg, 645 μg, 650 μg, 655 μg, 660 μg, 665 μg, 670 μg, 675 μg, 680 μg, 685 μg, 690 μg, 695 μg, 700 μg, or similar. It is clear to anyone skilled in the art that the suggested dosages per single dose specified above are not to be regarded as being limited to the numerical values actually stated. Fluctuations of about ±2.5 μg, particularly in the decimal range, are also included, as will be apparent to one of skill in the art. In these dosage ranges, the active substances 1e′ and 2 may be present in the weight ratios given above.

For example, without restricting the scope of the invention thereto, the combinations of 1e and 2 according to the invention may contain a quantity of cation 1e′ and 2 (values based on free base) such that, for each single dose, 8.2 μg of 1e′ and 5 μg of 2, 8.2 μg of 1e′ and 10 μg of 2, 8.2 μg of 1e′ and 15 μg of 2, 8.2 μg of 1e′ and 25 μg of 2, 8.2 μg of 1e′ and 50 μg of 2, 8.2 μg of 1e′ and 100 μg of 2, 16.5 μg of 1e′ and 5 μg of 2, 16.5 μg of 1e′ and 10 μg of 2, 16.5 μg of 1e′ and 15 μg of 2, 16.5 μg of 1e′ and 25 μg of 2, 16.5 μg of 1e′ and 50 μg of 2, 16.5 μg of 1e′ and 100 μg of 2, 33.0 μg of 1e′ and 5 μg of 2, 33.0 μg of 1e′ and 10 μg of 2, 33.0 μg of 1e′ and 15 μg of 2, 33.0 μg of 1e′ and 25 μg of 2, 33.0 μg of 1e′ and 50 μg of 2, 33.0 μg of 1e′ and 100 μg of 2, 49.5 μg of 1e′ and 5 μg of 2, 49.5 μg of 1e′ and 10 μg of 2, 49.5 μg of 1e′ and 15 μg of 2, 49.5 μg of 1e′ and 25 μg of 2, 49.5 μg of 1e′ and 50 μg of 2, 49.5 μg of 1e′ and 100 μg of 2, 82.5 μg of 1e′ and 5 μg of 2, 82.5 μg of 1e′ and 10 μg of 2, 82.5 μg of 1e′ and 15 μg of 2, 82.5 μg of 1e′ and 25 μg of 2, 82.5 μg of 1e′ and 50 μg of 2, 82.5 μg of 1e′ and 100 μg of 2, 165.0 μg of 1e′ and 5 μg of 2, 165.0 μg of 1e′ and 10 μg of 2, 165.0 μg of 1e′ and 15 μg of 2, 165.0 μg of 1e′and 25 μg of 2, 165.0 μg of 1e′ and 50 μg of 2, 165.0 μg of 1e′ and 100 μg of 2, 206.2 μg of 1e′ and 5 μg of 2, 206.2 μg of 1e′ and 10 μg of 2, 206.2 μg of 1e′ and 15 μg of 2, 206.2 μg of 1e′ and 25 μg of 2, 206.2 μg of 1e′ and 50 μg of 2, 206.2 μg of 1e′ and 100 μg of 2, 412.5 μg of 1e′ and 5 μg of 2, 412.5 μg of 1e′ and 10 μg of 2, 412.5 μg of 1e′ and 15 μg of 2, 412.5 μg of 1e′ and 25 μg of 2, 412.5 μg of 1e′ and 50 μg of 2, and 412.5 μg of 1e′ and 100 μg of 2 are present, for example.

From the aforementioned examples for suitable doses of the 1e′ containing combinations according to the invention, the corresponding amounts of the salts 1e and of the acid addition salts of 2 are readily calculable.

For compositions according to the invention that contain as the anticholinergic a compound of formula 1f or 1g the weight ratios and amounts of 1f/1 and 2 are in the range of those suggested hereinbefore for combinations containing 1e and 2.

The aforementioned examples of possible doses applicable for the combinations according to the invention are to be understood as referring to doses per single application. However, these examples are not be understood as excluding the possibility of administering the combinations according to the invention multiple times. Depending on the medical need patients may receive also multiple inhalative applications. As an example, patients may receive the combinations according to the invention for instance two or three times (e.g., two or three puffs with a powder inhaler, an MDI, etc.) in the morning of each treatment day. As the aforementioned dose-examples are only to be understood as dose examples per single application (i.e., per puff) multiple application of the combinations according to the invention leads to multiple doses of the aforementioned examples. The application of the compositions according to the invention can be for instance once a day, or depending on the duration of action of the anticholinergic agent twice a day, or once every 2 or 3 days.

Moreover, it is emphasized that the aforementioned dose examples are to be understood as examples of metered doses only. In other terms, the aforementioned dose examples are not to be understood as the effective doses of the combinations according to the invention that do in fact reach the lung. It is clear for the person of ordinary skill in the art that the delivered dose to the lung is generally lower than the metered dose of the administered active ingredients.

The active substance combinations of 1 and 2 according to the invention are preferably administered by inhalation. For this purpose, ingredients 1 and 2 have to be made available in forms suitable for inhalation. Inhalable preparations according to the invention include inhalable powders, propellant-containing metered dose aerosols, or propellant-free inhalable solutions. Inhalable powders according to the invention containing the combination of active substances 1 and 2 may consist of the active substances on their own or of a mixture of the active substances with physiologically acceptable excipients. Within the scope of the present invention, the term carrier may optionally be used instead of the term excipient. Within the scope of the present invention, the term propellant-free inhalable solutions also includes concentrates or sterile inhalable solutions ready for use. The preparations according to the invention may contain the combination of active substances 1 and 2 either together in one formulation or in two separate formulations. These formulations which may be used within the scope of the present invention are described in more detail in the next part of the specification.

A. Inhalable Powder Containing the Combinations of Active Substances 1 and 2 According to the Invention

The inhalable powders according to the invention may contain 1 and 2 either on their own or in admixture with suitable physiologically acceptable excipients. If the active substances 1 and 2 are present in admixture with physiologically acceptable excipients, the following physiologically acceptable excipients may be used to prepare these inhalable powders according to the invention: monosaccharides (e.g., glucose or arabinose), disaccharides (e.g., lactose, saccharose, maltose, or trehalose), oligo- and polysaccharides (e.g., dextran), polyalcohols (e.g., sorbitol, mannitol, or xylitol), cyclodextrins (e.g., α-cyclodextrin, β-cyclodextrin, χ-cyclodextrin, methyl-β-cyclodextrin, hydroxypropyl-β-cyclodextrin), salts (e.g., sodium chloride or calcium carbonate) or mixtures of these excipients with one another. Preferably, mono- or disaccharides are used, while the use of lactose, trehalose, or glucose is preferred, particularly, but not exclusively, in the form of their hydrates.

Within the scope of the inhalable powders according to the invention the excipients have a maximum average particle size of up to 250 μm, preferably between 10 μm and 150 μm, most preferably between 15 μm and 80 μm. It may sometimes seem appropriate to add finer excipient fractions with an average particle size of 1 μm to 9 μm to the excipients mentioned above. These finer excipients are also selected from the group of possible excipients listed hereinbefore. Finally, in order to prepare the inhalable powders according to the invention, micronized active substance 1 and 2, preferably with an average particle size of 0.5 μm to 10 μm, more preferably from 1 μm to 6 μm, is added to the excipient mixture. Processes for producing the inhalable powders according to the invention by grinding and micronizing and by finally mixing the ingredients together are known from the prior art. The inhalable powders according to the invention may be prepared and administered either in the form of a single powder mixture which contains both 1 and 2 or in the form of separate inhalable powders which contain only 1 or 2.

The inhalable powders according to the invention may be administered using inhalers known from the prior art. Inhalable powders according to the invention which contain a physiologically acceptable excipient in addition to 1 and 2 may be administered, for example, by means of inhalers which deliver a single dose from a supply using a measuring chamber as described in U.S. Pat. No. 4,570,630, which is hereby incorporated by reference, or by other means as described in DE 36 25 685 A. The inhalable powders according to the invention which contain 1 and 2 optionally in conjunction with a physiologically acceptable excipient may be administered for example using an inhaler known by the name TURBUHALER® or using inhalers as disclosed, for example, in EP 237507 A. Preferably, the inhalable powders according to the invention which contain physiologically acceptable excipient in addition to 1 and 2 are packed into capsules (to produce so-called inhalettes) which are used in inhalers as described, for example, in WO 94/28958 (corresponding to U.S. Pat. No. 5,947,118, which is hereby incorporated by reference).

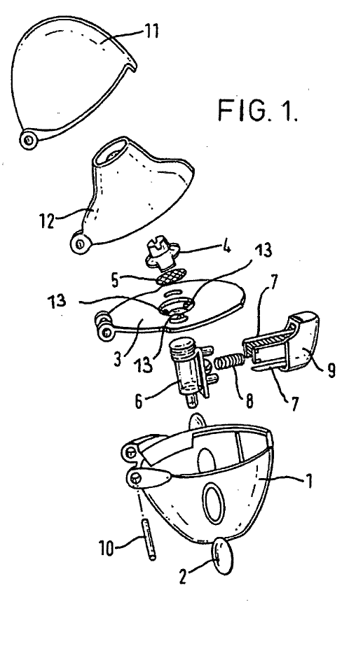

A particularly preferred inhaler for administering the pharmaceutical combination according to the invention in inhalettes is shown in FIG. 1.

The inhaler according to FIG. 1 is characterized by a housing 1 containing two windows 2, a deck 3 in which there are air inlet ports and which is provided with a screen 5 secured via a screen housing 4, an inhalation chamber 6 connected to the deck 3 on which there is a push button 9 provided with two sharpened pins 7 and movable counter to a spring 8, a mouthpiece 12 which is connected to the housing 1, the deck 3 and a cover 11 via a spindle 10 to enable it to be flipped open or shut and three holes 13 with diameters below 1 mm in the central region around the capsule chamber 6 and underneath the screen housing 4 and screen 5.

The main air flow enters the inhaler between deck 3 and base 1 near to the hinge. The deck has in this range a reduced width, which forms the entrance slit for the air. Then the flow reverses and enters the capsule chamber 6 through the inlet tube. The flow is then further conducted through the filter and filter holder to the mouthpiece. A small portion of the flow enters the device between mouthpiece and deck and flows then between filter holder and deck into the main stream. Due to production tolerances, there is some uncertainty in this flow because of the actual width of the slit between filter holder and deck. In case of new or reworked tools, the flow resistance of the inhaler may therefore be a little off the target value. To correct this deviation, the deck has in the central region around the capsule chamber 6 and underneath the screen housing 4 and screen 5 three holes 13 with diameters below 1 mm. Through these holes 13 flows air from the base into the main air stream and reduces such slightly the flow resistance of the inhaler. The actual diameter of these holes 13 can be chosen by proper inserts in the tools so that the mean flow resistance can be made equal to the target value.

If the inhalable powders according to the invention are packed into capsules (inhalettes) for the preferred use described above, the quantities packed into each capsule should be 1 mg to 30 mg per capsule. These capsules contain, according to the invention, either together or separately, the doses of 1 and 2 mentioned hereinbefore for each single dose.

B. Propellant Gas-Driven Inhalation Aerosols Containing the Combinations of Active Substances 1 and 2

Inhalation aerosols containing propellant gas according to the invention may contain substances 1 and 2 dissolved in the propellant gas or in dispersed form. 1 and 2 may be present in separate formulations or in a single preparation, in which 1 and 2 are either both dissolved, both dispersed or only one component is dissolved and the other is dispersed. The propellant gases which may be used to prepare the inhalation aerosols according to the invention are known from the prior art. Suitable propellant gases are selected from among hydrocarbons such as n-propane, n-butane, or isobutane and halohydrocarbons such as fluorinated derivatives of methane, ethane, propane, butane, cyclopropane, or cyclobutane. The propellant gases mentioned above may be used on their own or in mixtures thereof. Particularly preferred propellant gases are halogenated alkane derivatives selected from TG11, TG12, TG134a (1,1,1,2-tetrafluoroethane), and TG227 (1,1,1,2,3,3,3-heptafluoropropane), and mixtures thereof, of which the propellant gases TG134a, TG227, and mixtures thereof are preferred.

The propellant-driven inhalation aerosols according to the invention may also contain other ingredients such as cosolvents, stabilizers, surfactants, antioxidants, lubricants, and pH adjusters. All these ingredients are known in the art.

The inhalation aerosols containing propellant gas according to the invention may contain up to 5 wt.-% of active substance 1 and/or 2. Aerosols according to the invention contain, for example, 0.002 to 5 wt.-%, 0.01 to 3 wt.-%, 0.015 to 2 wt.-%, 0.1 to 2 wt.-%, 0.5 to 2 wt.-%, or 0.5 to 1 wt.-% of active substance 1 and/or 2.

If the active substances 1 and/or 2 are present in dispersed form, the particles of active substance preferably have an average particle size of up to 10 μm, preferably from 0.1 μm to 6 μm, more preferably from 1 μm to 5 μm.

The propellant-driven inhalation aerosols according to the invention mentioned above may be administered using metered dose inhalers (MDIs) known in the art.

Accordingly, in another aspect, the present invention relates to pharmaceutical compositions in the form of propellant-driven aerosols as hereinbefore described combined with one or more inhalers suitable for administering these aerosols. In addition, the present invention relates to inhalers which are characterized in that they contain the propellant gas-containing aerosols described above according to the invention. The present invention also relates to cartridges fitted with a suitable valve which can be used in a suitable inhaler and which contain one of the abovementioned propellant gas-containing inhalation aerosols according to the invention. Suitable cartridges and methods of filling these cartridges with the inhalable aerosols containing propellant gas according to the invention are known from the prior art.

C. Propellant-Free Inhalable Solutions or Suspensions Containing the Combinations of Active Substances 1 and 2 According to the Invention

Propellant-free inhalable solutions and suspensions according to the invention contain, for example, aqueous or alcoholic, preferably ethanolic solvents, optionally ethanolic solvents mixed with aqueous solvents. If aqueous/ethanolic solvent mixtures are used the relative proportion of ethanol compared with water is not limited but preferably the maximum is up to 70 percent by volume, more particularly up to 60 percent by volume of ethanol. The remainder of the volume is made up of water. The solutions or suspensions containing 1 and 2, separately or together, are adjusted to a pH of 2 to 7, preferably 2 to 5, using suitable acids. The pH may be adjusted using acids selected from inorganic or organic acids. Examples of particularly suitable inorganic acids include hydrochloric acid, hydrobromic acid, nitric acid, sulfuric acid, and/or phosphoric acid. Examples of particularly suitable organic acids include ascorbic acid, citric acid, malic acid, tartaric acid, maleic acid, succinic acid, fumaric acid, acetic acid, formic acid, and/or propionic acid, etc. Preferred inorganic acids are hydrochloric and sulfuric acids. It is also possible to use the acids which have already formed an acid addition salt with one of the active substances. Of the organic acids, ascorbic acid, fumaric acid, and citric acid are preferred. If desired, mixtures of the above acids may be used, particularly in the case of acids which have other properties in addition to their acidifying qualities, e.g., as flavorings, antioxidants, or complexing agents, such as citric acid or ascorbic acid, for example. According to the invention, it is particularly preferred to use hydrochloric acid to adjust the pH.

According to the invention, the addition of edetic acid (EDTA) or one of the known salts thereof, sodium edetate, as stabilizer or complexing agent is unnecessary in the present formulation. Other embodiments may contain this compound or these compounds. In a preferred embodiment the content based on sodium edetate is less than 100 mg/100 mL, preferably less than 50 mg/100 mL, more preferably less than 20 mg/100 mL. Generally, inhalable solutions in which the content of sodium edetate is from 0 to 10 mg/100 mL are preferred.

Cosolvents and/or other excipients may be added to the propellant-free inhalable solutions which may be used according to the invention. Preferred cosolvents are those which contain hydroxyl groups or other polar groups, e.g., alcohols, particularly isopropyl alcohol, glycols, particularly propyleneglycol, polyethyleneglycol, polypropylene glycol, glycol ether, and glycerol, and polyoxyethylene alcohols and polyoxyethylene fatty acid esters. The terms excipients and additives in this context denote any pharmacologically acceptable substance which is not an active substance but which can be formulated with the active substance or substances in the pharmacologically suitable solvent in order to improve the qualitative properties of the active substance formulation. Preferably, these substances have no pharmacological effect or, in connection with the desired therapy, no appreciable or at least no undesirable pharmacological effect. The excipients and additives include, for example, surfactants such as soya lecithin, oleic acid, sorbitan esters, such as polysorbates, polyvinylpyrrolidone, other stabilizers, complexing agents, antioxidants, and/or preservatives which guarantee or prolong the shelf life of the finished pharmaceutical formulation, flavorings, vitamins and/or other additives known in the art. The additives also include pharmacologically acceptable salts such as sodium chloride as isotonic agents.

The preferred excipients include antioxidants such as ascorbic acid, for example, provided that it has not already been used to adjust the pH, vitamin A, vitamin E, tocopherols, and similar vitamins and provitamins occurring in the human body.

Preservatives may be used to protect the formulation from contamination with pathogens. Suitable preservatives are those which are known in the art, particularly cetyl pyridinium chloride, benzalkonium chloride, or benzoic acid or benzoates such as sodium benzoate in the concentration known from the prior art. The preservatives mentioned above are preferably present in concentrations of up to 50 mg/100 mL, more preferably between 5 and 20 mg/100 mL.

Preferred formulations contain, in addition to the solvent water and the combination of active substances 1 and 2, only benzalkonium chloride and sodium edetate. In another preferred embodiment, no sodium edetate is present.

The propellant-free inhalable solutions which may be used within the scope of the invention are administered in particular using inhalers of the kind which are capable of nebulizing a small amount of a liquid formulation in the therapeutic dose within a few seconds to produce an aerosol suitable for therapeutic inhalation. Within the scope of the present invention, preferred inhalers are those in which a quantity of less than 100 μL, preferably less than 50 μL, more preferably between 10 μL and 30 μL of active substance solution can be nebulized in preferably one spray action to form an aerosol with an average particle size of less than 20 μm, preferably less than 10 μm, in such a way that the inhalable part of the aerosol corresponds to the therapeutically effective quantity.

An apparatus of this kind for propellant-free delivery of a metered quantity of a liquid pharmaceutical composition for inhalation is described for example in International Patent Application WO 91/14468 (corresponding to U.S. Pat. No. 5,497,944, which is hereby incorporated by reference) and also in WO 97/12687 (corresponding to U.S. Pat. No. 5,964,416, which is hereby incorporated by reference) (cf in particular FIGS. 6a and 6b). The nebulizers (devices) described therein are also known by the name RESPIMAT®.

This RESPIMAT® nebulizer can advantageously be used to produce the inhalable aerosols according to the invention containing the combination of the active substances 1 and 2. Because of its cylindrical shape and handy size of less than 9 cm to 15 cm long and 2 cm to 4 cm wide, this device can be carried at all times by the patient. The nebulizer sprays a defmed volume of pharmaceutical formulation using high pressures through small nozzles so as to produce inhalable aerosols.

The preferred atomizer essentially consists of an upper housing part, a pump housing, a nozzle, a locking mechanism, a spring housing, a spring, and a storage container, characterized by:
a pump housing which is secured in the upper housing part and which comprises at one end a nozzle body with the nozzle or nozzle arrangement,
a hollow plunger with valve body,
a power takeoff flange in which the hollow plunger is secured and which is located in the upper housing part,
a locking mechanism situated in the upper housing part,
a spring housing with the spring contained therein, which is rotatably mounted on the upper housing part by means of a rotary bearing, and
a lower housing part which is fitted onto the spring housing in the axial direction.

The hollow plunger with valve body corresponds to a device disclosed in WO 97/12687 (corresponding to U.S. Pat. No. 5,964,416). It projects partially into the cylinder of the pump housing and is axially movable within the cylinder. Reference is made in particular to FIGS. 1 to 4, especially FIG. 3, and the relevant parts of the description. The hollow plunger with valve body exerts a pressure of 5 MPa to 60 MPa (about 50 bar to 600 bar), preferably 10 MPa to 60 MPa (about 100 bar to 600 bar) on the fluid, the measured amount of active substance solution, at its high pressure end at the moment when the spring is actuated. Volumes of 10 to 50 microliters are preferred, while volumes of 10 to 20 microliters are particularly preferred and a volume of 15 microliters per spray is most particularly preferred.

The valve body is preferably mounted at the end of the hollow plunger facing the valve body.

The nozzle in the nozzle body is preferably microstructured, i.e., produced by microtechnology. Microstructured valve bodies are disclosed, for example, in WO 94/07607 (corresponding to U.S. Pat. No. 5,911,851, which is hereby incorporated by reference); reference is hereby made to the contents of this specification, particularly FIG. 1 therein and the associated description.

The nozzle body consists, for example, of two sheets of glass and/or silicon firmly joined together, at least one of which has one or more microstructured channels which connect the nozzle inlet end to the nozzle outlet end. At the nozzle outlet end there is at least one round or non-round opening 2 to 10 microns deep and 5 to 15 microns wide, the depth preferably being 4.5 to 6.5 microns while the length is preferably 7 to 9 microns.

In the case of a plurality of nozzle openings, preferably two, the directions of spraying of the nozzles in the nozzle body may extend parallel to one another or may be inclined relative to one another in the direction of the nozzle opening. In a nozzle body with at least two nozzle openings at the outlet end the directions of spraying may be at an angle of 20° to 160° to one another, preferably 60° to 150°, most preferably 80° to 100°. The nozzle openings are preferably arranged at a spacing of 10 to 200 microns, more preferably at a spacing of 10 to 100 microns, most preferably 30 to 70 microns. Spacings of 50 microns are most preferred. The directions of spraying will therefore meet in the vicinity of the nozzle openings.

The liquid pharmaceutical preparation strikes the nozzle body with an entry pressure of up to 600 bar, preferably 200 bar to 300 bar, and is atomized into an inhalable aerosol through the nozzle openings. The preferred particle or droplet sizes of the aerosol are up to 20 microns, preferably 3 to 16 microns.

The locking mechanism contains a spring, preferably a cylindrical helical compression spring, as a store for the mechanical energy. The spring acts on the power takeoff flange as an actuating member the movement of which is determined by the position of a locking member. The travel of the power takeoff flange is precisely limited by an upper and lower stop. The spring is preferably biased, via a power step-up gear, e.g., a helical thrust gear, by an external torque which is produced when the upper housing part is rotated counter to the spring housing in the lower housing part. In this case, the upper housing part and the power takeoff flange have a single or multiple V-shaped gear.

The locking member with engaging locking surfaces is arranged in a ring around the power takeoff flange. It consists, for example, of a ring of plastic or metal which is inherently radially elastically deformable. The ring is arranged in a plane at right angles to the atomizer axis. After the biasing of the spring, the locking surfaces of the locking member move into the path of the power takeoff flange and prevent the spring from relaxing. The locking member is actuated by means of a button. The actuating button is connected or coupled to the locking member. In order to actuate the locking mechanism, the actuating button is moved parallel to the annular plane, preferably into the atomizer; this causes the deformable ring to deform in the annual plane. Details of the construction of the locking mechanism are given in WO 97/20590 (corresponding to U.S. Pat. No. 6,453,795, which is hereby incorporated by reference).

The lower housing part is pushed axially over the spring housing and covers the mounting, the drive of the spindle and the storage container for the fluid.

When the atomizer is actuated, the upper housing part is rotated relative to the lower housing part, the lower housing part taking the spring housing with it. The spring is thereby compressed and biased by means of the helical thrust gear and the locking mechanism engages automatically. The angle of rotation is preferably a whole-number fraction of 360°, e.g., 180°. At the same time as the spring is biased, the power takeoff part in the upper housing part is moved along by a given distance, the hollow plunger is withdrawn inside the cylinder in the pump housing, as a result of which some of the fluid is sucked out of the storage container and into the high pressure chamber in front of the nozzle.

If desired, a number of exchangeable storage containers which contain the fluid to be atomized may be pushed into the atomizer one after another and used in succession. The storage container contains the aqueous aerosol preparation according to the invention.

The atomizing process is initiated by pressing gently on the actuating button. As a result, the locking mechanism opens up the path for the power takeoff member. The biased spring pushes the plunger into the cylinder of the pump housing. The fluid leaves the nozzle of the atomizer in atomized form.

Further details of construction are disclosed in PCT Applications WO 97/12683 (corresponding to U.S. Pat. No. 6,176,442, which is hereby incorporated by reference) and WO 97/20590 (corresponding to U.S. Pat. No. 6,176,442), to which reference is hereby made.

The components of the atomizer (nebulizer) are made of a material which is suitable for its purpose. The housing of the atomizer and, if its operation permits, other parts as well are preferably made of plastics, e.g., by injection molding. For medicinal purposes, physiologically safe materials are used.

FIGS. 6a/b of WO 97/12687 show the RESPIMAT® nebulizer which can advantageously be used for inhaling the aqueous aerosol preparations according to the invention.

FIG. 6a (WO 97/12687) shows a longitudinal section through the atomizer with the spring biased while FIG. 6b (WO 97/12687) shows a longitudinal section through the atomizer with the spring relaxed.

The upper housing part (51) contains the pump housing (52) on the end of which is mounted the holder (53) for the atomizer nozzle. In the holder is the nozzle body (54) and a filter (55). The hollow plunger (57) fixed in the power takeoff flange (56) of the locking mechanism projects partially into the cylinder of the pump housing. At its end, the hollow plunger carries the valve body (58). The hollow plunger is sealed off by means of the seal (59). Inside the upper housing part is the stop (60) on which the power takeoff flange abuts when the spring is relaxed. On the power takeoff flange is the stop (61) on which the power takeoff flange abuts when the spring is biased. After the biasing of the spring, the locking member (62) moves between the stop (61) and a support (63) in the upper housing part. The actuating button (64) is connected to the locking member. The upper housing part ends in the mouthpiece (65) and is sealed off by means of the protective cover (66) which can be placed thereon.

The spring housing (67) with compression spring (68) is rotatably mounted on the upper housing part by means of the snap-in lugs (69) and rotary bearing. The lower housing part (70) is pushed over the spring housing. Inside the spring housing is the exchangeable storage container (71) for the fluid (72) which is to be atomized. The storage container is sealed off by the stopper (73) through which the hollow plunger projects into the storage container and is immersed at its end in the fluid (supply of active substance solution). The spindle (74) for the mechanical counter is mounted in the covering of the spring housing. At the end of the spindle facing the upper housing part is the drive pinion (75). The slider (76) sits on the spindle.

The nebulizer described above is suitable for nebulizing the aerosol preparations which may be used according to the invention to produce an aerosol suitable for inhalation.

If the formulation according to the invention are nebulized using the method described above (RESPIMAT® nebulizer) the quantity delivered should correspond to a defined quantity with a tolerance of not more than 25%, preferably 20% of this amount in at least 97%, preferably at least 98% of all operations of the inhaler (spray actuations). Preferably, between 5 mg and 30 mg of formulation, most preferably between 5 mg and 20 mg of formulation are delivered as a defined mass on each actuation.

However, the formulation according to the invention may also be nebulized by means of inhalers other than those described above, e.g., jet stream inhalers or other stationary nebulizers.

Accordingly, in a fuirther aspect, the invention relates to the method according to the invention administering pharmaceutical formulations in the form of propellant-free inhalable solutions or suspensions as described above combined with a device suitable for administering these formulations, preferably in conjunction with the RESPIMAT® nebulizer. Preferably, the invention relates to propellant-free inhalable solutions or suspensions characterized by the combination of active substances 1 and 2 according to the invention in conjunction with the RESPIMAT® nebulizer. In addition, the present invention relates to the use according to the invention of the above-mentioned devices for inhalation, preferably the RESPIMAT® nebulizer, characterized in that they contain the propellant-free inhalable solutions or suspensions according to the invention as described hereinbefore.

According to the invention, inhalable solutions which contain the active substances 1 and 2 in a single preparation are preferred. The term “single preparation” also includes preparations which contain the two ingredients 1 and 2 in two-chamber cartridges, as disclosed, for example, in WO 00/23037 (corresponding to U.S. Pat. No. 6,481,435, which is hereby incorporated by reference).

The propellant-free inhalable solutions or suspensions which may be used within the scope of the invention may take the form of concentrates or sterile inhalable solutions or suspensions ready for use, as well as the abovementioned solutions and suspensions designed for use in a RESPIMAT® nebulizer. Formulations ready for use may be produced from the concentrates, for example, by the addition of isotonic saline solutions. Sterile formulations ready for use may be administered using energy-operated fixed or portable nebulizers which produce inhalable aerosols by means of ultrasound or compressed air by the Venturi principle or other principles.

Accordingly, in another aspect, the present invention relates to pharmaceutical compositions in the form of propellant-free inhalable solutions or suspensions as described hereinbefore which take the form of concentrates or sterile formulations ready for use, combined with a device suitable for administering these solutions, characterized in that the device is an energy-operated free-standing or portable nebulizer which produces inhalable aerosols by means of ultrasound or compressed air by the Venturi principle or other methods.

The Examples which follow serve to illustrate the present invention in more detail without restricting the scope of the invention to the following embodiments by way of example. First, the preparation of exemplified compounds 1e, 1f, and 1g which are not known in the art will be described.

I. Examples and Preparation of the Compounds of Formula 1e

### EXAMPLE 1

### tropenol 9-hydroxyfluorene-9-carboxylate methobromide

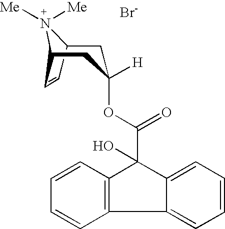 1.1: methyl 9-hydroxyfluorene-9-carboxylate

50.4 g (0.223 mol) of 9-hydroxy-9-fluorenecarboxylic acid is dissolved in 500 mL of methanol, combined with 5 mL (0.089 mol) of concentrated sulfuric acid, and refluxed for 1 hour. After cooling, 100 mL of sodium hydrogen carbonate solution (about pH 8) is added and the methanol is largely evaporated down. The mixture is extracted with dichloromethane and water, and the organic phase is dried and evaporated to dryness. The product is purified by recrystallization from ethyl acetate. Yield: 50.0 g of white crystals (93% of theory).

1.2: tropenol 9-hydroxyfluorene-9-carboxylate

13.4 g (0.056 mol) of methylester 1.1, 11.65 g (0.084 mol) of tropenol, and 0.3 g of sodium are heated as a melt at 75 mbar for 4 hours over a bath of boiling water with occasional agitation. After cooling, the sodium residues are dissolved with acetonitrile, the solution is evaporated to dryness, and the residue is extracted with dichloromethane-water. The organic phase is washed with water, dried over MgSO₄, and the solvent is distilled off. The product is purified by recrystallization from diethyl ether. Yield: 11.40 g of white crystals (29% of theory).

1.3: tropenol 9-hydroxyfluorene-9-carboxylate methobromide

1.75 g (0.005 mol) of compound 1.2 is taken up in 30 mL dichloromethane and 15 mL acetonitrile and combined with 2.85 g (0.015 mol) of 50% methylbromide solution in acetonitrile. The reaction mixture is left to stand for 3 days at ambient temperature, during which time the product crystallizes. The crystals precipitated are separated off and recrystallized from diethyl ether to purify them. Yield: 1.95 g of white crystals (88% of theory); melting point: 250° C.; elemental analysis: calculated: C, (62.45); H, (5.47); N, (3.17). found: C, (61.53); H, (5.84); N, (3.22).

### EXAMPLE 2

### tropenol 9-fluorofluorene-9-carboxylate methobromide

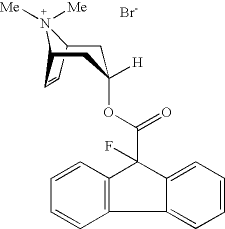 2.1: tropenol 9-fluorofluorene-9-carboxylate

1.66 mL (0.009 mol) of bis(2-methoxyethyl)aminosulfur trifluoride is placed in 10 mL dichloromethane and within 20 minutes at 15° C. to 20° C., a solution of 2.4 g (0.007 mol) of compound 1.2 in 25 mL of dichloromethane is added dropwise thereto. The mixture is stirred for 20 hours at ambient temperature, cooled to 0° C. and carefully combined with 80 mL of water with thorough stirring. Then the mixture is carefully adjusted to pH 8 with aqueous NaHCO₃ solution, the organic phase is separated off, the aqueous phase is extracted again with dichloromethane, the combined organic phases are washed with water, dried over MgSO₄, and evaporated to dryness. The hydrochloride is precipitated and recrystallized from acetonitrile-diethyl ether. Then the free base is liberated again using 10% aqueous sodium carbonate solution. Yield: 1.05 g of bright yellow crystals (53% of theory).

2.2: tropenol 9-fluorofluorene-9-carboxylate methobromide

1.05 g (0.003 mol) of compound 2.1 is taken up in 20 mL acetonitrile and reacted with 1.71 g (0.009 mol) of 50% methyl bromide solution in acetonitrile analogously to step 1.3. To purify it, the product is recrystallized from acetonitrile. Yield: 0.80 g of white crystals (60% of theory); melting point: 252° C.; elemental analysis: calculated: C, (62.17); H, (5.22); N, (3.15). found: C, (62.04); H, (5.23); N, (3.15).

### EXAMPLE 3

### scopine 9-hydroxyfluorene-9-carboxylate methobromide

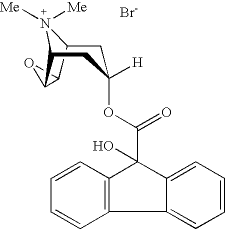 3.1: scopine 9-hydroxyfluorene-9-carboxylate

9.0 g (0.026 mol) of tropenol ester 2.1 are suspended in 90 mL of dimethylformamide and combined with 0.47 g (0.003 mol) of vanadium (V) oxide. At 60° C., a solution of 4.89 g (0.052 mol) of H₂O₂-urea in 20 mL of water is added dropwise and stirred for 6 hours at 60° C. After cooling to 20° C., the precipitate formed is suction filtered, the filtrate is adjusted to pH 2 with 4 N hydrochloric acid and combined with Na₂S₂O₅ dissolved in water. The resulting solution is evaporated to dryness, and the residue is extracted with dichloromethane-water. The acidic aqueous phase is made basic with Na₂CO₃, extracted with dichloromethane, and the organic phase is dried over Na₂SO₄ and concentrated. Then 1 mL of acetyl chloride is added at ambient temperature and the mixture is stirred for 1 hour. After extraction with 1 N hydrochloric acid, the aqueous phase is made basic, extracted with dichloromethane, and the organic phase is washed with water and dried over Na₂SO₄. Then the solvent is removed by distillation. The crude product is purified by recrystallization from diethyl ether. Yield: 2.8 g of white crystals (30% of theory).

3.2: scopine 9-hvdroxyfluorene-9-carboxylate methobromide

1.3 g (0.004 mol) of compound 3.1 is taken up in 20 mL of chloroform and 20 mL of acetonitrile and reacted with 2.279 g (0.012 mol) of 50% methylbromide solution in acetonitrile analogously to step 1.3. To purify it, the product is recrystallized from acetonitrile. Yield: 1.25 g of light beige crystals (68% of theory); melting point: 243° C.-244° C.; elemental analysis: calculated: C, (60.27); H, (5.28); N, (3.06). found: C, (60.03); H, (5.35); N, (3.55).

### EXAMPLE 4

### scopine 9-fluorofluorene-9-carboxylate methobromide

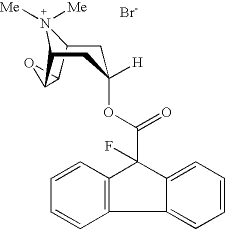 4.1: scopine 9-fluorofluorene-9-carboxylate

0.885 mL (0.005 mol) of bis-(2-methoxyethyl)aminosulfur trifluoride is placed in 25 mL of dichloromethane and reacted with 1.42 g (0.004 mol) of compound 3.1 analogously to the procedure according to 2.1. Yield: 1.1 g of beige crystals (75% of theory).

4.2: scopine 9-fluorofluorene-9-carboxylate methobromide

1.1 g (0.003 mol) of compound 4.1 is taken up in 30 mL acetonitrile and reacted with 1.71 g (0.009 mol) of 50% methyl bromide solution in acetonitrile analogously to step 1.3. To purify it, the product is recrystallized from isopropanol. Yield: 0.45 g of white crystals (33% of theory); melting point: 200° C.-201° C.; elemental analysis: calculated: C, (60.01); H, (5.04); N, (3.04). found: C, (59.91); H, (5.18); N, (3.10).

### EXAMPLE 5

### tropenol 9-methvlfluorene-9-carboxylate methobromide

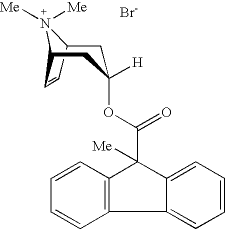 5.1: 9-methylfluorene-9-carboxylic acid

a) methyl 9-methylfluorene-9-carboxylate

From 7.6 g (0.33 mol) of sodium and 300 mL of ethanol, a sodium ethoxide solution is prepared, to which 69.6 g (0.33 mol) of 9-fluorenecarboxylic acid is added batchwise. After the addition has ended, the mixture is stirred for 2.5 hours at ambient temperature. Then it is evaporated to dryness, and the residue is suspended in 600 mL of dimethylformamide and 93.96 g (0.662 mol) of methyl iodide are added dropwise. The mixture is stirred for 3 hours at constant temperature. The cloudy solution is stirred into 500 mL of water and 300 mL of diethyl ether with cooling and extracted, the organic phase is washed with water and 10% sodium carbonate solution, dried and evaporated to dryness. The residue is purified by column chromatography, eluent: cyclohexane-ethyl acetate (96:4). Yield: 12.61 g of white crystals (16% of theory); melting point: 108° C.-109° C.

b) 9-methylfluorene-9-carboxylic acid

12.6 g (0.053 mol) of methyl 9-methylfluorene-9-carboxylate and 53 mL of 2 molar aqueous sodium hydroxide solution are stirred in 120 mL of 1,4-dioxane for 24 hours at ambient temperature. The dioxane is distilled off, made up to a total volume of 300 mL with water, and extracted with diethyl ether. The aqueous phase is acidified with 3 molar aqueous HCl, crystallized, and filtered. Yield: 11.25 g of white crystals (95% of theory); melting point: 168° C.-169° C.

5.2: tropenol 9-methylfluorene-9-carboxylate

6.73 g (0.03 mol) of compound 5.1 is suspended in 60 mL dichloromethane, combined with 5.0 g of oxalyl chloride, and 1 drop of dimethylformamide, then stirred for one hour at ambient temperature and finally the solvent is distilled off. The acid chloride remaining is used in the next step without any further purification.

4.18 g (0.03 mol) of tropenol and 4.27 g (0.033 mol) of diisopropylethylamine are suspended in 100 mL of dichloroethane, and the acid chloride is added dropwise to 30 mL of dichloroethane at 35° C.-40° C. and then stirred for 24 hours at 40° C. The suspension is diluted with dichloromethane and extracted with dilute hydrochloric acid. The organic phase is then washed with water, dried over MgSO₄, and the product is converted into its hydrochloride with a solution of HCl in diethyl ether. The solvent is then removed. To purify it, the precipitated hydrochloride is taken up in water and extracted with diethyl ether. The aqueous phase is made basic with 10% aqueous sodium carbonate solution and extracted with dichloromethane. The organic phase is dried over MgSO₄ and the solvent is distilled off. Yield: 4.40 g of yellow oil (42% of theory).

5.3: tropenol 9-methylfluorene-9-carboxylate methobromide

1.8 g (0.005 mol) of the free base 5.2 is reacted analogously to the method in step 1.3. The product is purified by recrystallization from acetone. Yield: 1.80 g of white crystals (82% of theory); melting point: 258° C.-259° C.; elemental analysis: calculated: C, (65.46); H, (5.95); N, (3.18). found: C, (64.15); H, (5.95); N, (3.18).

### EXAMPLE 6

### scopine 9-methvlfluorene-9-carboxylate methobromide

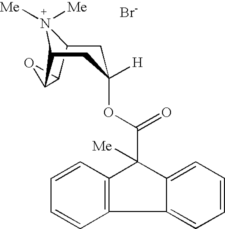 6.1: scopine 9-methylfluorene-9-carboxylate

2.5 g (0.007 mol) of tropenol ester 5.2 is reacted with 0.13 g (0.001 mol) of vanadium (V) oxide and 1.43 g (0.015 mol) of H₂O₂-urea analogously to the process according to step 3.1. Yield: 1.8 g of white crystals (71% of theory).

6.2: scopine 9-methylfluorene-9-carboxylate methobromide

1.8 g (0.005 mol) of 6.1 is taken up in 30 mL acetonitrile and reacted with 2.848 g (0.015 mol) of 50% methyl bromide solution in acetonitrile analogously to step 1.3. Yield: 1.6 g of white crystals (70% of theory); melting point: 214° C.; elemental analysis: calculated: C, (62.13); H, (5.93); N, (4.26). found: C, (62.23); H, (6.05); N, (4.32).

II. Examples and Preparation of the Compounds of Formula 1f

Preparation of the Cyclopropyltropine Starting Material

35 mL (0.35 mol) of 40% aqueous potassium hydroxide solution is overlaid with 100 mL of diethyl ether and cooled in the ice bath. For this, 23.64 g (0.101 mol) of N-methyl-N-nitrosourea are added batchwise and then the mixture is stirred for about 10 minutes. The ether phase is decanted off and the solution obtained is used in the following step.

25 mL of the diazomethane solution prepared above is added to a solution of 4.01 g (0.028 mol) of tropenol in 25 mL of diethyl ether and 5 mL of methanol while cooling with an ice bath. Then 53.4 mg (0.000139 mol) of bis(benzonitrile)dichloropalladium (II) is added. A further 28 mL of the diazomethane solution are then added batchwise. After about 1.5 hours, the solvent is distilled off in vacuo, the residue remaining is extracted, and this solution is filtered and the solvent is removed by distillation. Yield: 4.25 g of slightly yellowish crystals (96% of theory).

### EXAMPLE 7

### cvclopropyltropine benzilate methobromide

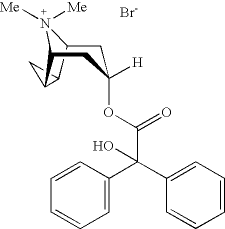 7.1: methylbenzilate

90 g (0.394 mol) of benzilic acid is dissolved in 900 mL acetonitrile and, at 5° C., 109.6 g (0.72 mol) of DBU are added dropwise. After the addition of 204.4 g (1.44 mol) of methyl iodide, the mixture is stirred for 24 hours at ambient temperature (about 20° C.-23° C.). The solution is evaporated down to the residue, the residue is taken up in diethyl ether and extracted with water. The organic phase is washed with 5% aqueous sodium carbonate solution and water, dried, and the solvent is distilled off. The product is purified by recrystallization from cyclohexane. Yield: 77.19 g of white crystals (81% of theory); melting point: 74° C.-76° C.

7.2: cyclopropyltropinebenzilate

5.34 g (0.022 mol) of methylbenzilate 7.1, 1.53 g (0.01 mol) of cyclopropyltropine, and 0.25 g (0.01 mol) of sodium are heated as a melt over a bath of boiling water at 75 mbar for 1 hour with occasional shaking. After cooling, the sodium residues are dissolved with acetonitrile, the solution is evaporated to dryness, and the residue is extracted with dichloromethane/water. The organic phase is extracted with 10% potassium hydrogen sulfate solution, and the resulting aqueous phase is made basic and extracted with dichloromethane. The organic phase is separated off, dried, and evaporated to dryness. The product is purified by recrystallization from acetonitrile. Yield: 2.41 g of white crystals (66% of theory).

7.3: cyclopropyltropine benzilate methobromide

0.46 g (0.0013 mol) of compound 7.2 is taken up in 5 mL of acetonitrile and stirred with 1.53 g (0.0082 mol) of 50% methyl bromide solution in acetonitrile in a pressurised reaction vessel at 80° C. After 2 days, the solution is evaporated to dryness, and the residue is taken up in acetonitrile and filtered while hot. After cooling, the precipitated crystals are separated off, dried, and recrystallized from acetonitrile. Yield: 0.066 g of white crystals (11% of theory); melting point: 208° C.-209° C.; elemental analysis: calculated: C, (62.89); H, (6.16); N, (3.06). found: C, (62.98); H, (6.20); N, (3.03).

### EXAMPLE 8

### cyclopropyltropine 2,2-diphenylpropionate methobromide

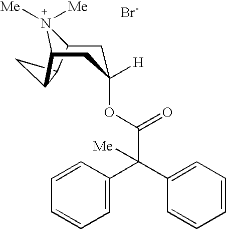 8.1: 2,2-diphenylpropionic acid chloride

52.08 g (0.33 mol) of oxalyl chloride is slowly added dropwise at 20° C. to a suspension of 25.0 g (0.11 mol) of 2,2-diphenylpropionic acid, 100 mL of dichloromethane, and 4 drops of dimethylformamide. It is stirred for 1 hour at 20° C. and 0.5 hour at 50° C. The solvent is distilled off and the residue remaining is used in the next step without any further purification.

8.2: cyclopropyltropine 2,2-diphenylpropionate

2.3 g (0.015 mol) of cyclopropyltropine and 2.13 g (0.016 mol) of diisopropylethylamine are placed in 30 mL of dichloromethane and within 15 minutes combined with a solution of acid chloride 8.1 in dichloromethane. Then the mixture is stirred for 2 hours at ambient temperature and 72 hours at 40° C. For working up, it is washed with water, dried over MgSO₄, and the solvent is distilled off. The product is converted into its hydrochloride with a solution of HCl in diethyl ether. To purify it, the precipitated hydrochloride is taken up in water and extracted with diethyl ether. The aqueous phase is made basic with 10% aqueous sodium carbonate solution and extracted with dichloromethane. The organic phase is dried over MgSO₄ and the solvent is distilled off. Yield: 2.15 g of yellow oil (36% of theory)

8.3: Cyclopropyltropine 2,2-diphenylpropionate methobromide

1.8 g (0.005 mol) of the free base 8.2 are reacted analogously to the method in step 7.3. The purification is carried out by recrystallization from acetonitrile/diethyl ether. Yield: 1.53 g of white crystals (67% of theory); melting point: 208° C.-209° C.; elemental analysis: calculated: C, (65.79); H, (6.63); N, (3.07). found: C, (65.47); H, (6.77); N, (3.03).

### EXAMPLE 9

### cyclopropyltropine 9-hydroxyxanthene-9-carboxylate methobromide

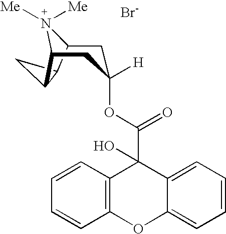 9.1: methyl 9-hydroxyxanthene-9-carboxylate

a) methyl xanthene-9-carboxylate

A sodium ethoxide solution is generated from 21.75 g (0.95 mol) of sodium and 1500 mL of ethanol. 214 g (0.95 mol) of xanthene-9-carboxylic acid is added batchwise to this solution and the resulting suspension is stirred for 1 hour at ambient temperature. Then the solid is separated off, washed with 1500 mL of diethyl ether, and the isolated crystals are suspended in 1500 mL of dimethylformamide and 126.73 mL (2.0 mol) of methyl iodide is added with stirring. The solution obtained is left to stand for 24 hours at ambient temperature, then diluted with water to a total volume of 6 L, crystallized, suction filtered, washed with water, and dried. Yield: 167 g of white crystals (74% of theory); melting point: 82° C.

b) methyl 9-hydroxyxanthene-9-carboxylate

48.05 g (0.2 mol) of methyl xanthene-9-carboxylate is dissolved in 1200 mL of tetrahydrofuran and combined with 23.63 g (0.2 mol) of potassium tert-butoxide at 0° C. Oxygen is then piped in for 2 hours at −10° C. to −5° C., then the mixture is acidified with 2 N aqueous hydrochloric acid and most of the solvent is removed by distillation. The residue remaining is extracted with ethyl acetate and water, and the organic phase is extracted with aqueous Na₂S₂O₅ solution, washed with water, dried, and the solvent is distilled off. The product is purified by crystallization from diisopropylether and cyclohexane. Yield: 11.10 g of white crystals (22% of theory)

9.2: cyclopropyltropine [9-hydroxyxanthene-9-carboxylate]

6.0 g (0.023 mol) of compound 9.1, 3.065 g (0.02 mol) of cyclopropyltropine, and 0.02 g of sodium are reacted analogously to step 7.2. Yield: 2.2 g of white crystals (25% of theory); melting point: 115° C.-116° C.

9.3: cyclopropyltropine 9-hydroxyxanthene-9-carboxylate methobromide

2.1 g (0.006 mol) of the free base 9.2 is reacted analogously to the method in step 7.3. The purification is carried out by recrystallization from isopropanol. Yield: 1.05 g of beige crystals (37% of theory); melting point: 218° C.; elemental analysis: calculated: C, (61.02); H, (5.55); N, (2.97). found: C, (60.40); H, (5.72); N, (2.96).

### EXAMPLE 10

### cyclopropyltropine 9-methylfluorene-9-carboxylate methobromide

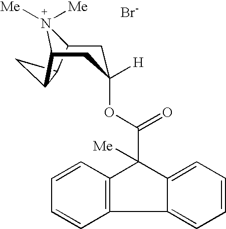 10.1: 9-methvlfluorene-9-carboxylic acid

a) methyl 9-methylfluorene-9-carboxylate

A sodium ethoxide solution is prepared from 7.6 g (0.33 mol) sodium and 300 mL of ethanol, and 69.6 g (0.33 mol) of 9-fluorenecarboxylic acid is added batchwise thereto. After the addition has ended, it is stirred for 2.5 hours at ambient temperature. Then it is evaporated to dryness, the residue is suspended in 600 mL of dimethylformamide and 93.96 g (0.662 mol) of methyl iodide is added dropwise. The mixture is stirred for 3 hours at constant temperature. The cloudy solution is stirred into 500 mL of water and 300 mL of diethyl ether with cooling, extracted, and the organic phase is washed with water and 10% sodium carbonate solution, dried, and evaporated to dryness. The residue is purified by column chromatography, eluent: cyclohexane-ethyl acetate (96:4). Yield: 12.61 g of white crystals (16% of theory); melting point: 108° C.-109° C.

b) 9-methylfluorene-9-carboxylic acid

12.6 g (0.053 mol) of methyl 9-methylfluorene-9-carboxylate and 53 mL of 2 molar aqueous sodium hydroxide solution are stirred in 120 mL of 1,4-dioxane for 24 hours at ambient temperature. The dioxane is distilled off, water is added to give a total volume of 300 mL, and the mixture is extracted with diethyl ether. The aqueous phase is acidified with 3 molar aqueous HCl, crystallized, and filtered. Yield: 11.25 g of white crystals (95% of theory); melting point: 168° C.-169° C.

10.2: cyclopropyltropine 9-methylfluorene-9-carboxylate

The acid chloride is prepared from 4.0 g (0.018 mol) of compound 10.1, 4.53 g (0.036 mol) of oxalyl chloride, and 4 drops of dimethylformamide in 40 mL dichloromethane. 2.48 g (0.016 mol) of cyclopropyltropine and 1.91 g (0.019-mol) of triethylamine are suspended in 30 mL of dichloroethane, and the acid chloride is added dropwise to 30 mL of dichloroethane at 30° C. within 15 minutes and then stirred for 24 hours at 40° C. The suspension is extracted with dichloromethane and water, the organic phase is washed with aqueous acetic acid, dried, and the solvent is removed by distillation. The product is converted into its hydrochloride. To purify it, the precipitated hydrochloride is taken up in water and extracted with diethyl ether. The aqueous phase is made basic and extracted with dichloromethane. The organic phase is dried over MgSO₄ and the solvent is distilled off. The crude product is purified by recrystallization from acetonitrile. Yield: 1.81 g of slightly beige crystals (30% of theory); melting point: 138° C.-139° C.

10.3: cyclopropyltropine 9-methylfluorene-9-carboxylate methobromide

1.81 g (0.005 mol) of the free base 10.2 is reacted analogously to the method in step 7.3. The purification is carried out by recrystallization from acetonitrile. Yield: 1.26 g of white crystals (56% of theory); melting point: 228° C.-229° C.; elemental analysis: calculated: C, (66.09); H, (6.21); N, (3.08). found: C, (66.26); H, (6.26); N, (3.11).

### EXAMPLE 11

### cyclopropyltropine 9-methylxanthene-9-carboxylate methobromide

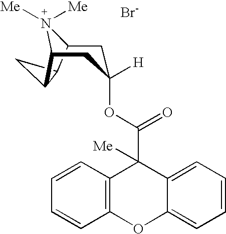 11.1: 9-methylxanthene-9-carboxylic acid

a) methyl 9-methylxanthene-9-carboxylate

Starting from 9.61 g (0.04 mol) of methyl 9-xanthenecarboxylate (obtainable according to step 9.1.a), the reaction to obtain the title compound is carried out analogously to the method in step 10.1.a. Yield: 6.05 g of white crystals (60% of theory); melting point: 91° C.-92° C.

b) 9-methylxanthene-9-carboxylic acid

Starting from 20.34 g (0.08 mol) of methyl 9-methylxanthene-9-carboxylate, the reaction to obtain the title compound is carried out analogously to the method in step 10.1.b. Yield: 14.15 g of white crystals (74% of theory); melting point: 207° C.-208° C.

11.2 Cyclopropyltropine 9-methylxanthene-9-carboxylate

The acid chloride is prepared from 5.0 g (0.021 mol) of compound 11.1, 5.53 g (0.042 mol) of oxalyl chloride, and 4 drops of dimethylformamide in 50 mL of dichloromethane. 3.06 g (0.02 mol) of cyclopropyltropine and the acid chloride produced above are reacted analogously to the method in step 10.2 to obtain the title compound. Yield: 1.95 g of slightly beige crystals (26% of theory); melting point: 87° C.-88° C.

11.3: cyclopropyltropine 9-methylxanthene-9-carboxylate methobromide

1.95 g (0.005 mol) of the free base 11.1 is reacted analogously to the method in step 7.3. The purification is carried out by recrystallization from acetonitrile. Yield: 0.54 g of white crystals (23% of theory); melting point: 193° C.-194° C.; elemental analysis: calculated: C, (63.83); H, (6.00); N, (2.98). found: C, (61.42); H, (6.24); N, (2.97).

### EXAMPLE 12

### cyclopropyltropine 9-hydroxyfluorene-9-carboxylate methobromide

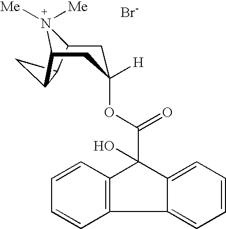 12.1: methyl 9-hydroxyfluorene-9-carboxylate

50.4 g (0.223 mol) of 9-hydroxy-9-fluorenecarboxylic acid is dissolved in 500 mL of methanol, combined with 5 mL (0.089 mol) of concentrated sulfuric acid and refluxed for 1 hour. After cooling, 100 mL of sodium hydrogen carbonate solution (approximately pH 8) is added and the methanol is largely evaporated down. It is extracted with dichloromethane and water, and the organic phase is dried and evaporated to dryness. The purification is carried out by recrystallization from ethyl acetate. Yield: 50.0 g of white crystals (93% of theory).

12.2: cyclopropyltropine 9-hydroxyfluorene-9-carboxylate

6.0 g (0.025 mol) of compound 12.1, 3.45 g (0.023 mol) of cyclopropyltropine, and 0.03 g of sodium are reacted analogously to step 7.2. The purification is carried out by recrystallization from acetonitrile. Yield: 3.46 g of white crystals (38% of theory); melting point: 131° C.-132° C.

12.3: cyclopropyltropine 9-hydroxyfluorene-9-carboxylate methobromide

3.36 g (0.009 mol) of the free base 12.2 is reacted analogously to the method in step 7.3. The purification is carried out by recrystallization from isopropanol. Yield: 3.32 g of white crystals (79% of theory); melting point: 219° C.-220° C.; elemental analysis: calculated: C, (63.16); H, (5.74); N, (3.07). found: C, (62.93); H, (5.93); N, (3.10).

### EXAMPLE 13

### cyclopropyltropine 4,4′-difluoromethyl benzilate methobromide

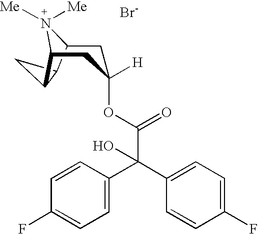 13.1: 4.4′-difluoromethyl benzilate

a) 4,4′-difluorobenzilic acid

A solution of 24.62 g (0.1 mol) of 4,4′-difluorobenzil in 250 mL dioxane is added dropwise to a solution of 49.99 g (1.25 mol) of NaOH flakes in 300 mL of water at about 100° C. and stirred for 2 hours. The dioxane is largely distilled off and the aqueous solution remaining is extracted with dichloromethane. When the aqueous solution is acidified with sulfuric acid, a precipitate is deposited, which is suction filtered, washed, and dried. The filtrate is extracted with dichloromethane, and the organic phase is dried over Na₂SO₄ and evaporated to dryness. Yield: 25.01 g (95% of theory); melting point: 133° C.-136° C.

b) 4,4′-difluoromethyl benzilate

25.0 g (0.095 mol) of 4,4′-difluorobenzilic acid is added to freshly prepared sodium ethoxide solution from 2.17 g (0.095 mol) of sodium and 200 mL of ethanol at 20° C. and stirred for 3 hours. The solution is evaporated to dryness, the residue is dissolved in DMF, and 22.57 g (0.16 mol) of methyl iodide is added dropwise at 20° C. and the mixture is stirred for 24 hours. 300 mL of water is then added dropwise to the suspension formed, while cooling with ice, and the mixture is extracted with diethyl ether and the organic phase is washed with water, dried over Na₂SO₄, and evaporated to dryness. Yield: 21.06 g (80% of theory).

13.2: cyclopropyltropine 4,4′-difluoromethyl benzilate

6.2 g (0.022 mol) of compound 13.1, 3.37 g (0.022 mol) of cyclopropyltropine, and 0.051 g of sodium are reacted analogously to step 7.2 to obtain the product. The purification is carried out by recrystallization from acetonitrile. Yield: 4.15 g of white crystals (47% of theory); melting point: 120° C.-121 ° C.

13.3: cyclopropyltropine 4,4′-difluoromethyl benzilate methobromide

2.0 g (0.005 mol) of the free base 13.2 is reacted analogously to the method in step 7.3. The purification is carried out by recrystallization from ethanol-diethyl ether. Yield: 1.8 g of white crystals (73% of theory); melting point: 206° C.-207° C.; elemental analysis: calculated: C, (58.31); H, (5.30); N, (2.83). found: C, (58.15); H, (5.42); N, (2.84).

III. Examples and Preparation of the Compounds of Formula 1g

### EXAMPLE 14

### tropenol 9-hydroxyxanthene-9-carboxylate methobromide

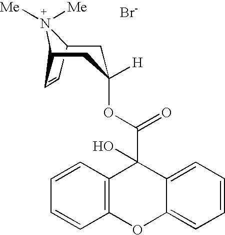 14.1: methyl 9-hydroxyxanthene-9-carboxylate

a) methylxanthene-9-carboxylate

A sodium ethoxide solution is prepared from 21.75 g (0.95 mol) of sodium and 1500 mL of ethanol. 214 g (0.95 mol) of xanthene-9-carboxylic acid is added batchwise to this solution and the suspension obtained is stirred for 1 hour at ambient temperature. Then the solid is separated off, washed with 1500 mL diethyl ether, and the isolated crystals are suspended in 1500 mL of dimethylformamide and combined with 126.73 mL (2.0 mol) of methyl iodide with stirring. The solution formed is left to stand for 24 hours at ambient temperature, then diluted with water to a total volume of 6 L, crystallized, suction filtered, washed with water, and dried. Yield: 167 g of white crystals (74% of theory); melting point: 82° C.

b) methyl 9-hydroxyxanthene-9-carboxylate

48.05 g (0.2 mol) methylxanthene-9-carboxylate is dissolved in 1200 mL of tetrahydrofuran and, at 0° C., combined with 23.63 g (0.2 mol) of potassium tert-butoxide. Oxygen is the piped in for 2 hours at −10° C. to −5° C., then the mixture is acidified with 2 N aqueous hydrochloric acid, and the majority of the solvent is distilled off. The residue remaining is extracted with ethyl acetate and water, and the organic phase is extracted with aqueous Na₂S₂O₅ solution, washed with water, dried, and the solvent is distilled off. The product is purified by crystallization from diisopropylether and cyclohexane. Yield: 11.10 g of white crystals (22% of theory)

14.2: tropenol 9-hydroxyxanthene-9-carboxylate

13.65 g (0.053 mol) of methylester 14.1, 8.35 g (0.06 mol) of tropenol, and 0.2 g of sodium are heated as a melt at 75 mbar for 4 hours over a bath of boiling water with occasional agitation. After cooling, the sodium residues are dissolved with acetonitrile, the solution is evaporated to dryness, and the residue is extracted with dichloromethane-water. The organic phase is washed with water, dried over MgSO₄, and the solvent is distilled off. The product is purified by recrystallization from diethyl ether-petroleum ether. Yield: 5.28 g of white crystals (27% of theory); melting point: 117° C.

14.3: tropenol 9-hydroxyxanthene-9-carboxylate methobromide

0.8 g (0.002 mol) of compound 14.2 is taken up in 20 mL of dichloromethane and 20 mL of acetonitrile and combined with 1.14 g (0.006 mol) of 50% methylbromide solution in acetonitrile. The reaction mixture is left to stand for 3 days at ambient temperature, during which time the product crystallizes. The crystals precipitated are separated off and recrystallized from acetone to purify them. Yield: 0.94 g of white crystals (93% of theory); melting point: 249° C.-250° C.; elemental analysis: calculated: C, (60.27); H, (5.28); N, (3.06). found: C, (60.04); H, (5.34); N, (2.98).

### EXAMPLE 15

### scopine 9-hydroxyxanthene-9-carboxylate methobromide

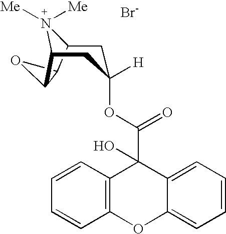 15.1: scopine 9-hydroxyxanthene-9-carboxylate

6.8 g (0.019 mol) of tropenol ester 14.2 is suspended in 75 mL of dimethylformamide and combined with 0.36 g (0.002 mol) of vanadium (V) oxide. At 60° C., a solution of 3.52 g (0.037 mol) of H₂O₂-urea in 15 mL of water is added dropwise and the mixture is stirred for 6 hours at 60° C. After cooling to 20° C., the mixture is adjusted to pH 2 with 4 N hydrochloric acid and combined with Na₂S₂O₅ dissolved in water. The resulting solution is evaporated to dryness, and the residue is extracted with dichloromethane-water. The acidic aqueous phase obtained is made basic with Na₂CO₃, extracted with dichloromethane and the organic phase is dried over Na₂SO₄ and concentrated. Then 1 mL of acetyl chloride is added at ambient temperature and the mixture is stirred for 1 hour. After extraction with 1 N hydrochloric acid, the aqueous phase is made basic, extracted with dichloromethane, and the organic phase is washed with water and dried over MgSO₄. Finally, the solvent is distilled off. The crude product is purified by recrystallization from diethyl ether. Yield: 5.7 g of yellow oil (79% of theory).

15.2: scopine 9-hydroxyxanthene-9-carboxylate methobromide

4.0 g (0.011 mol) of compound 15.1 is taken up in 60 mL acetonitrile and reacted with 6.27 g (0.033 mol) of 50% methyl bromide solution in acetonitrile analogously to step 14.3. Yield: 3.6 g of white crystals (69% of theory); melting point: 226° C.-227° C.; elemental analysis: calculated: C, (58.24); H, (5.10); N, (2.95). found: C, (58.33); H, (4.98); N, (3.05).

### EXAMPLE 16

### tropenol 9-methylxanthene-9-carboxylate methobromide

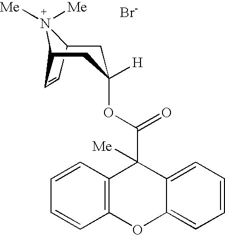 16.1: 9-methylxanthene-9-carboxylic acid

a) methyl 9-methylxanthene-9-carboxylate

9.61 g (0.04 mol) of methyl 9-xanthenecarboxylate (obtainable according to step 14.1.a) is dissolved in 150 mL of tetrahydrofuran, combined with a solution of 5.0 g (0.042 mol) potassium tert-butoxide in THF, and stirred for 10 minutes. 5 mL (0.08 mol) of methyl iodide is then added dropwise with gentle cooling and, after all has been added, the mixture is stirred for 1 hour at ambient temperature. The reaction mixture is diluted with water to a total volume of 800 mL, extracted with diethyl ether, and the organic phase is extracted with saturated, aqueous Na₂CO₃ solution, washed with water, dried over MgSO₄, and the solvent is distilled off. The product is purified by recrystallization from methanol. Yield: 6.05 g of white crystals (70% of theory); melting point: 91° C.-92° C.

b) 9-methylxanthene-9-carboxylic acid

20.34 g (0.08 mol) of the methyl ester described above and 80 mL of 2 molar aqueous sodium hydroxide solution are stirred in 200 mL dioxane for 24 hours at ambient temperature, then the dioxane is distilled off, the mixture is made up to a total volume of 600 mL with water, extracted with diethyl ether, and the aqueous phase is acidified with 4 N hydrochloric acid. The product crystallizes, and is suction filtered and washed with water. It is purified by recrystallization from acetonitrile. Yield: 14.15 g of white crystals (74% of theory); melting point: 207° C.-208° C.

16.2: tropenol 9-methylxanthene-9-carboxylate

From 7.76 g (0.03 mol) of compound 16.1, 0.06 mol of oxalyl chloride, and 4 drops of dimethylformamide, the acid chloride is prepared in 100 mL dichloromethane. It is added dropwise as a solution in dichloromethane to 8.77 g (0.063 mol) of tropenol in 140 mL of dichloromethane, then stirred for 24 hours at 40° C. and cooled. The reaction mixture is extracted with water, dried over MgSO₄, and filtered off. The filtrate obtained is acidified to pH 2 with ethereal hydrochloric acid, extracted with diethyl ether, and the aqueous phase is made basic. After extraction with dichloromethane, the organic phase is washed neutral with water, dried over MgSO₄, and evaporated to dryness: The residue is dissolved in diethyl ether, insoluble matter is filtered off, and the solvent is removed by distillation. Yield: 3.65 g of yellow oil (34% of theory).

16.3: tropenol 9-methylxanthene-9-carboxylate methobromide

1.65 g (0.005 mol) of compound 16.2 is taken up in 20 mL acetonitrile and reacted with 2.85 g (0.015 mol) of 50% methyl bromide solution in acetonitrile analogously to step 14.3. Yield: 1.5 g of white crystals (65% of theory); melting point: 212° C.-213° C.; elemental analysis: calculated: C, (63.16); H, (5.74); N, (3.07). found: C, (62.50); H, (5.94); N, (3.11).

### EXAMPLE 17

### scopine 9-methylxanthene-9-carboxylate methobromide

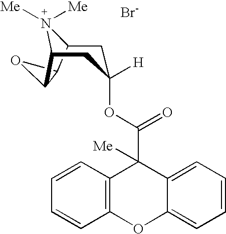 17.1: scopine 9-methylxanthene-9-carboxylate

1.9 g (0.005 mol) of tropenol ester 16.2 is suspended in 30 mL of dimethylformamide and reacted with 0.12 g (0.001 mol) of vanadium (V) oxide and 0.01 mol of H₂O₂-urea in water analogously to the method according to step 15.1. Yield: 1.4 g of white crystals (74% of theory).

17.2: scopine 9-methylxanthene-9-carboxylate methobromide

1.35 g (0.004 mol) of compound 17.1 is taken up in 10 mL of dichloromethane and 20 mL of acetonitrile and reacted with 2.28 g (0.012 mol) of 50% methyl bromide solution in acetonitrile analogously to step 14.3. Yield: 1.35 g of white crystals (71% of theory); melting point: 208° C.-209° C.; elemental analysis: calculated: C, (61.02); H, (5.55); N, (2.97); found: C, (59.78); H, (5.70); N, (2.96).

### EXAMPLE 18

### tropenol 9-ethylxanthene-9-carboxylate methobromide

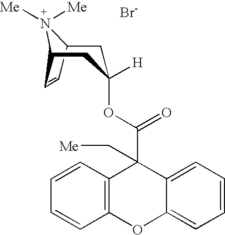 18.1: 9-ethylxanthene-9-carboxylic acid

a) methyl 9-ethylxanthene-9-carboxylate

10.0 g (0.042 mol) of methyl 9-xanthenecarboxylate (obtainable according to step 14.1.a) is dissolved in 100 mL of tetrahydrofuran and combined batchwise with 5.16 g (0.044 mol) of potassium tert-butoxide while cooling. Then, at about 18° C.-22° C., 6.296 mL (0.083 mol) of bromoethane is added dropwise and, after it has all been added, the mixture is stirred for about 1.5 hours at ambient temperature. The precipitate formed is suction filtered and the solvent is removed by distillation. The residue remaining is taken up in diethyl ether and extracted with water. The organic phase is dried over MgSO₄ and the solvent is removed by distillation. The crude product obtained is used in the next step without any further purification. Yield: 7.92 g of yellow oil (70% of theory).

b) 9-ethylxanthene-9-carboxylic acid

7.92 g (0.03 mol) of the ethyl ester described above and 29.5 mL of 2 molar aqueous sodium hydroxide solution are refluxed in 80 mL dioxane for 2.5 hours. The mixture is worked up as in step 3.1.b). Yield: 4.46 g of white crystals (58% of theory); melting point: 175° C.-176° C.

18.2: tropenol 9-ethylxanthene-9-carboxylate

From 4.46 g (0.03 mol) of compound 18.1, 4.45 g (0.035 mol) of oxalyl chloride, and 3 drops of dimethylformamide, the acid chloride is prepared in 40 mL of dichloromethane. It is added as a solution in dichloromethane to 4.87 g (0.035 mol) of tropenol in 60 mL of dichloromethane and reacted analogously to the procedure according to step 16.2 and worked up. Yield: 0.97 g of oil (15% of theory).

18.3: tropenol 9-ethylxanthene-9-carboxylate methobromide

0.97 g (0.003 mol) of compound 18.2 is taken up in 70 mL acetonitrile and reacted with 1.77 g (0.009 mol) of 50% methyl bromide solution in acetonitrile analogously to step 14.3. To purify it, the product is recrystallized from acetonitrile. Yield: 0.65 g of white crystals (46% of theory); melting point: 217° C.-218° C.; elemental analysis: calculated: C, (63.83); H, (6.00); N, (2.98). found: C, (61.76); H, (6.32); N, (2.92).

### EXAMPLE 19

### tropenol 9-difluoromethylxanthene-9-carboxylate methobromide

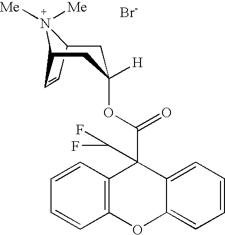 19.1: 9-difluoromethylxanthene-9-carboxylic acid

a) methyl 9-difluoromethylxanthene-9-carboxylate

16.8 g (0.07 mol) of methyl 9-xanthenecarboxylate (obtainable according to step 14.1 .a) is dissolved in 300 mL of tetrahydrofuran and 9.1 g (0.077 mol) of potassium tert-butoxide is added batchwise while cooling. Then difluorochloromethane is piped in at 0° C. over a period of 1.5 hours. After all the gas has been piped in, the reaction mixture is left to stand for 72 hours at ambient temperature. The reaction mixture is then diluted with water to a total volume of about 2000 mL, extracted with ethyl acetate, and the organic phase is separated off and washed with 5% aqueous sodium carbonate solution. After being extracted again with water, the organic phase is dried over MgSO₄ and the solvent is removed by distillation. The crude product is purified by chromatography on silica gel (eluent: cyclohexane-ethyl acetate (98:2)) or by recrystallization from cyclohexane. Yield: 5.35 g of white crystals (26% of theory); melting point: 101° C.

b) 9-difluoromethylxanthene-9-carboxylic acid

5.38 g (0.019 mol) of the ester described above and 18.5 mL of 2 molar aqueous sodium hydroxide solution are reacted in 60 mL of dioxane and worked up analogously to the reaction in step 16.1.b). Yield: 2.77 g of white crystals (53% of theory); melting point: 181° C.-182° C.

19.2: 9-difluoromethylxanthene-9-carboxylate tropenol

From 2.77 g (0.01 mol) of compound 19.1, 1 mL of oxalyl chloride, and 1 drop of dimethylformamide, the acid chloride is prepared. It is added to 2.78 g (0.02 mol) of tropenol in 50 mL of 1,2-dichloroethane and reacted and worked up analogously to step 16.2. Yield: 0.6 g of oil (15% of theory).

19.3: tropenol 9-difluoromethylxanthene-9-carboxylate methobromide

0.6 g (0.002 mol) of compound 19.2 is taken up in 20 mL acetonitrile and reacted with 1.14 g (0.006 mol) of 50% methyl bromide solution in acetonitrile analogously to step 14.3. Yield: 0.44 g of beige crystals (45% of theory); melting point: 227° C.-228° C.; elemental analysis: calculated: C, (58.55); H, (4.91); N, (2.84). found: C, (57.19); H, (5.11); N, (2.86).

### EXAMPLE 20

### scopine 9-hydroxymethylxanthene-9-carboxylate methobromide

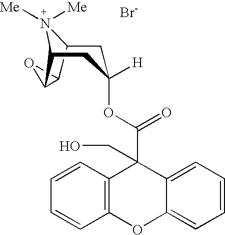 20.1: scopine 9-hydroxymethylxanthene-9-carboxylate

3.63 g (0.010 mol) of scopine xanthene-9-carboxylate, which may be obtained as described in WO 92/16528, is dissolved in 20 mL of dimethylformamide and combined with 0.36 g (0.012 mol) of paraformaldehyde. After the addition of 0.168 g (0.002 mol) of potassium tert-butoxide at 20° C., the mixture is stirred for 2 hours at ambient temperature. The mixture is acidified to pH 2 with 4 N hydrochloric acid with cooling and the solvent is distilled off in vacuo. The residue remaining is extracted with diethyl ether and water, and the aqueous phase is made basic with 10% sodium carbonate solution and extracted with dichloromethane. The organic phase is separated off and washed with water, dried, and the solvent is distilled off in vacuo. To purify it, the product is recrystallized from acetonitrile. Yield: 1.55 g white crystals (36% of theory); melting point: 232° C.

20.2: scopine 9-hydroxymethylxanthene-9-carboxylate methobromide

1.15 g (0.003 mol) of compound 20.1 is taken up in 20 mL acetonitrile and reacted with 1.71 g (0.009 mol) of 50% methyl bromide solution in acetonitrile analogously to step 14.3. Yield: 1.28 g of white crystals (87% of theory); melting point: 234° C.; elemental analysis: calculated: C, (59.02); H, (5.37); N, (2.87). found: C, (59.30); H, (5.41); N, (3.03).

IV. Examples of Formulations

The following examples of formulations, which may be obtained analogously to methods known in the art, serve to illustrate the present invention more fully without restricting it to the contents of these examples.

A. Inhalable Powders

| | μg per |
|---|---|
| Ingredients | capsule |
| 1) | |
| tiotropium bromide | 10.8 |
| 2 (hydrochloride) | 27.9 |
| lactose | 4961.3 |
| Total | 5000 |
| 2) | |
| tiotropium bromide | 21.7 |
| 2-en (hydrochloride) | 9.0 |
| lactose | 4969.3 |
| Total | 5000 |
| 3) | |
| tiotropium bromide × H₂O | 22.5 |
| 2-en (hydrochloride) | 18.0 |
| lactose | 4959.5 |
| Total | 5000 |
| 4) | |
| scopine 2,2-diphenylpropionic acid ester methobromide | 200 |
| 2-en (hydrochloride) | 12 |
| lactose | 24788 |
| Total | 25000 |
| 5) | |
| scopine 2,2-diphenylpropionic acid ester methobromide | 100 |
| 2 (hydrochloride) | 50 |
| lactose | 12350 |
| Total | 12500 |
| 6) | |
| scopine 2,2-diphenylpropionic acid ester methobromide | 50 |
| 2 (hydrochloride) | 50 |
| lactose | 4900 |
| Total | 5000 |
| 7) | |
| tropenol 2,2-diphenylpropionic acid ester methobromide | 200 |
| 2-en (hydrochloride) | 24 |
| lactose | 24776 |
| Total | 25000 |
| 8) | |
| scopine 3,3',4,4'-tetrafluorobenzilic acid ester methobromide | 100 |
| 2 (hydrochloride) | 50 |
| lactose | 12350 |
| Total | 12500 |
| 9) | |
| tropenol 3,3',4,4'-tetrafluorobenzilic acid ester methobromide | 100 |
| 2 (hydrochloride) | 50 |
| lactose | 12350 |
| Total | 12500 |
| 10) | |
| scopine 4,4'-tetrafluorobenzilic acid ester methobromide | 100 |
| 2 (hydrochloride) | 50 |
| lactose | 12350 |
| Total | 12500 |
| 11) | |
| tropenol 4,4'-tetrafluorobenzilic acid ester methobromide | 100 |
| 2 (hydrochloride) | 50 |
| lactose | 12350 |
| Total | 12500 |
| 12) | |
| 1a-en (bromide) | 150 |
| 2 (hydrochloride) | 50 |
| lactose | 12300 |
| Total | 12500 |
| 13) | |
| 1a-en (bromide) | 150 |
| 2-en (hydrochloride) | 50 |
| lactose | 12300 |
| Total | 12500 |
| 14) | |
| 1a-en (bromide) | 150 |
| 2-en (hydrochloride) | 15 |
| lactose | 12335 |
| Total | 12500 |
| 15) | |
| 1a-en (bromide) | 200 |
| 2 (hydrochloride) | 50 |
| lactose | 24750 |
| Total | 25000 |
| 16) | |
| Example 6 | 80 |
| 2-en (hydrochloride) | 12 |
| lactose | 12408 |
| Total | 12500 |
| 17) | |
| Example 6 | 30 |
| 2 (hydrochloride) | 50 |
| lactose | 12420 |
| Total | 12500 |
| 18) | |
| Example 9 | 80 |
| 2 (hydrochloride) | 50 |
| lactose | 12370 |
| Total | 12500 |
| 19) | |
| Example 6 | 100 |
| 2-en (hydrochloride) | 25 |
| lactose | 24875 |
| Total | 25000 |
| 20) | |
| Example 6 | 24 |
| 2-en (hydrochloride) | 12 |
| lactose | 4964 |
| Total | 5000 |

B. Propellant-Containing Inhalable Aerosols

| Ingredients | % by weight |
|---|---|
| 1) | |
| Example 6 | 0.010 |
| 2 (hydrochloride) | 0.066 |
| soya lecithin | 0.2 |
| TG134a:TG227 (2:3) | to 100 |
| 2) | |
| Example 6 | 0.030 |
| 2-en (hydrochloride) | 0.033 |
| absolute ethanol | 0.5 |
| isopropyl myristate | 0.1 |
| TG227 | to 100 |
| 3) | |
| Example 6 | 0.010 |
| 2-en (hydrochloride) | 0.035 |
| soya lecithin | 0.2 |
| TG134a:TG227 (2:3) | to 100 |
| 4) | |
| tiotropium bromide | 0.015 |
| 2 (hydrochloride) | 0.066 |
| soya lecithin | 0.2 |
| TG134a:TG227 (2:3) | to 100 |
| 5) | |
| tiotropium bromide | 0.029 |
| 2-en (hydrochloride) | 0.033 |
| absolute ethanol | 0.5 |
| isopropyl myristate | 0.1 |
| TG227 | to 100 |
| 6) | |
| tiotropium bromide | 0.042 |
| 2 (hydrochloride) | 0.047 |
| absolute ethanol | 30 |
| purified water | 1.5 |
| anhydrous citric acid | 0.002 |
| TG134a | to 100 |
| 7) | |
| scopine 2,2-diphenylpropionic acid ester methobromide | 0.020 |
| 2 (hydrochloride) | 0.066 |
| soya lecithin | 0.2 |
| TG11:TG12 (2:3) | to 100 |
| 8) | |
| scopine 2,2-diphenylpropionic acid ester methobromide | 0.039 |
| 2-en (hydrochloride) | 0.033 |
| absolute ethanol | 0.5 |
| Isopropyl myristate | 0.1 |
| TG227 | to 100 |
| 9) | |
| tropenol 2,2-diphenylpropionic acid ester methobromide | 0.020 |
| 2 (hydrochloride) | 0.066 |
| soya lecithin | 0.2 |
| TG11:TG12 (2:3) | to 100 |
| 10) | |
| tropenol 2,2-diphenylpropionic acid ester methobromide | 0.039 |
| 2-en (hydrochloride) | 0.033 |
| absolute ethanol | 0.5 |
| isopropyl myristate | 0.1 |
| TG227 | to 100 |
| 11) | |
| 1a-en (bromide) | 0.050 |
| 2 (hydrochloride) | 0.066 |
| soya lecithin | 0.2 |
| TG134a:TG227 (2:3) | to 100 |
| 12) | |
| 1a-en (bromide) | 0.080 |
| 2-en (hydrochloride) | 0.033 |
| absolute ethanol | 0.5 |
| isopropyl myristate | 0.1 |
| TG227 | to 100 |
| 13) | |
| 1a-en (bromide) | 0.050 |
| 2-en (hydrochloride) | 0.035 |
| soya lecithin | 0.2 |
| TG134a:TG227 (2:3) | to 100 |

1. A pharmaceutical composition comprising:
(a) a betamimetic of formula 2 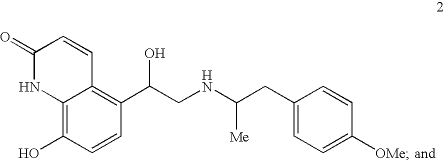
and
(b) an anticholinergic,
or an enantiomer, mixture of enantiomers, racemate, solvate, or hydrate thereof.

2. The pharmaceutical composition of claim 1, further comprising a pharmaceutically acceptable excipient.

3. The pharmaceutical composition of claim 1, wherein the betamimetic of formula 2 is the enantiomer of formula 2-en

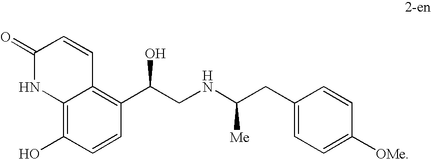

4. The pharmaceutical composition of claim 1, wherein the anticholinergic is a tiotropium salt, oxitropium salt, flutropium salt, ipratropium salt, glycopyrronium salt, or trospium salt, or an enantiomer, mixture of enantiomers, racemate, solvate, or hydrate thereof.

5. The pharmaceutical composition of claim 4, wherein the anticholinergic is a salt of chloride, bromide, iodide, sulfate, phosphate, methanesulfonate, nitrate, maleate, acetate, citrate, fumarate, tartrate, oxalate, succinate, benzoate or p-toluenesulfonate, or mixtures thereof.

6. The pharmaceutical composition of claim 1, wherein the anticholinergic is a tiotropium salt, oxitropium salt, or ipratropium salt, or an enantiomer, mixture of enantiomers, racemate, solvate, or hydrate thereof.

7. The pharmaceutical composition of claim 6, wherein the anticholinergic is a salt of chloride, bromide, iodide, sulfate, phosphate, methanesulfonate, nitrate, maleate, acetate, citrate, fumarate, tartrate, oxalate, succinate, benzoate or p-toluenesulfonate, or mixtures thereof.

8. The pharmaceutical composition of claim 1, wherein the anticholinergic is a salt of formula 1a 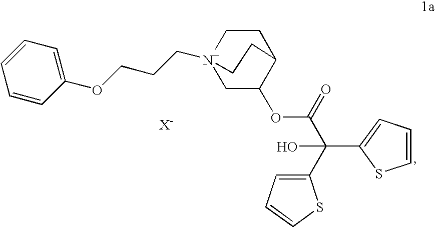
wherein X⁻ is an anion with a single negative charge,
or an enantiomer, racemate, hydrate, or mixture thereof.

9. The pharmaceutical composition of claim 8, wherein X⁻ is fluoride, chloride, bromide, iodide, sulfate, phosphate, methanesulfonate, nitrate, maleate, acetate, citrate, fumarate, tartrate, oxalate, succinate, benzoate, or p-toluenesulfonate, or an enantiomer, racemate, hydrate, or mixture thereof.

10. The pharmaceutical composition of claim 1, wherein the anticholinergic is a salt of formula 1a-en 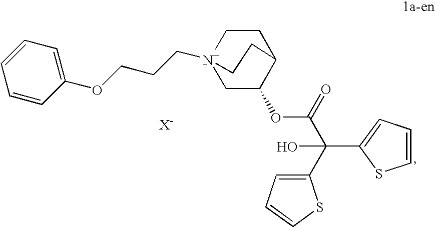
wherein X⁻ is an anion with a single negative charge,
or an enantiomer, racemate, hydrate, or mixture thereof.

11. The pharmaceutical composition of claim 10, wherein X⁻ is fluoride, chloride, bromide, iodide, sulfate, phosphate, methanesulfonate, nitrate, maleate, acetate, citrate, fumarate, tartrate, oxalate, succinate, benzoate, or p-toluenesulfonate, or an enantiomer, racemate, hydrate, or mixture thereof.

12. The pharmaceutical composition of claim 1, wherein the anticholinergic is a salt of formula 1b 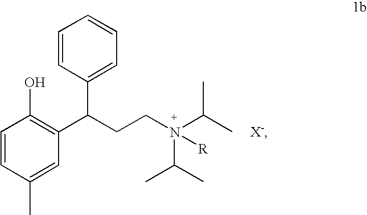
wherein:
R is methyl or ethyl; and
X⁻ is an anion with a single negative charge,
or an enantiomer, racemate, hydrate, or mixture thereof.

13. The pharmaceutical composition of claim 12, wherein X⁻ is fluoride, chloride, bromide, iodide, sulfate, phosphate, methanesulfonate, nitrate, maleate, acetate, citrate, fumarate, tartrate, oxalate, succinate, benzoate, or p-toluenesulfonate, or an enantiomer, racemate, hydrate, or mixture thereof.

14. The pharmaceutical composition according to claim 12, wherein the salt of formula 1b is an R-enantiomer.

15. The pharmaceutical composition of claim 1, wherein the anticholinergic is a compound of formula 1b-base 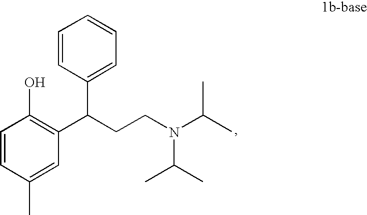
or an enantiomer, racemate, hydrate, or mixture thereof.

16. The pharmaceutical composition according to claim 12, wherein the compound of formula 1b-base is an R-enantiomer.

17. The pharmaceutical composition of claim 1, wherein the anticholinergic is a compound of formula 1c 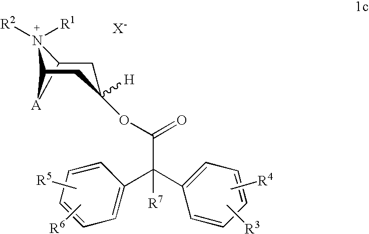
wherein: 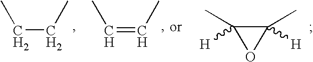
A is
X⁻ is an anion with a single negative charge;
R¹ and R², which are identical or different, are each methyl, ethyl, n-propyl, or isopropyl, each of which is optionally substituted by hydroxy or fluorine;
R³, R⁴, R⁵, and R⁶, which are identical or different, are each hydrogen, methyl, ethyl, methyloxy, ethyloxy, hydroxy, fluorine, chlorine, bromine, —CN, —CF₃, or —NO₂; and
R⁷ is hydrogen, methyl, ethyl, methyloxy, ethyloxy, —CH₂F, —CH₂CH₂F, —OCH₂F, —OCH₂CH₂F, —CH₂OH, —CH₂CH₂OH, —CF₃, —CH₂OMe, —CH₂CH₂OMe, —CH₂OEt, —CH₂CH₂OEt, —O—COMe, —O—COEt, —O—COCF₃, —O—COCF₃, fluorine, chlorine, or bromine,
or an enantiomer, racemate, hydrate, or mixture thereof.

18. The pharmaceutical composition of claim 17, wherein X⁻ is fluoride, chloride, bromide, iodide, sulfate, phosphate, methanesulfonate, nitrate, maleate, acetate, citrate, fumarate, tartrate, oxalate, succinate, benzoate, or p-toluenesulfonate, or an enantiomer, racemate, hydrate, or mixture thereof.

19. The pharmaceutical composition of claim 1, wherein the anticholinergic is a compound of formula 1d 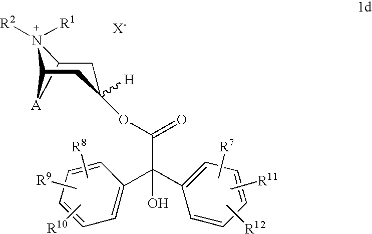
wherein:
A is 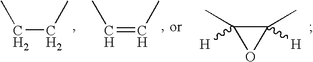
X⁻ is an anion with a single negative charge;
R¹ and R², which are identical or different, are each methyl, ethyl, n-propyl, and isopropyl, each optionally substituted by hydroxy or fluorine; and
R⁷, R⁸, R⁹, R¹⁰, R¹¹, and R¹², which are identical or different, are each hydrogen, methyl, ethyl, methyloxy, ethyloxy, hydroxy, fluorine, chlorine, bromine, —CN, —CF₃, or —NO₂, with the proviso that at least one of R⁷, R⁸, R⁹, R¹⁰, R¹¹, and R¹² is not hydrogen,
or an enantiomer, racemate, hydrate, or mixture thereof.

20. The pharmaceutical composition of claim 19, wherein X⁻ is fluoride, chloride, bromide, iodide, sulfate, phosphate, methanesulfonate, nitrate, maleate, acetate, citrate, fumarate, tartrate, oxalate, succinate, benzoate, or p-toluenesulfonate, or an enantiomer, racemate, hydrate, or mixture thereof.

21. The pharmaceutical composition of claim 1, wherein the anticholinergic is a compound of formula 1e 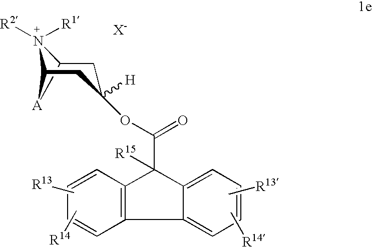
wherein: 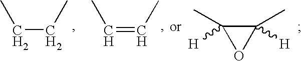
A is
X⁻ is an anion with a single negative charge;
R¹⁵ is hydrogen, hydroxy, methyl, ethyl, —CF₃, —CHF₂, or fluorine;
R^{1′} and R^{2′}, which are identical or different, are each C₁-C₅-alkyl optionally substituted by C₃-C₆-cycloalkyl, hydroxy, or halogen, or R^{1′} and R^{2′} together are a —C₃-C₅-alkylene-bridge; and
R¹³, R¹⁴, R^{13′}, and R^{14′}, which are identical or different, are each hydrogen, —C₁-C₄-alkyl, —C₁-C₄-alkyloxy, hydroxy, —CF₃, —CHF₂, —CN, —NO₂, or halogen,
or an enantiomer, racemate, hydrate, or mixture thereof.

22. The pharmaceutical composition of claim 21, wherein X⁻ is fluoride, chloride, bromide, iodide, sulfate, phosphate, methanesulfonate, nitrate, maleate, acetate, citrate, fumarate, tartrate, oxalate, succinate, benzoate, or p-toluenesulfonate, or an enantiomer, racemate, hydrate, or mixture thereof.

23. The pharmaceutical composition of claim 1, wherein the anticholinergic is a compound of formula 1f 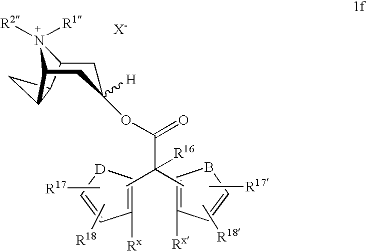
wherein:
X⁻ is an anion with a single negative charge;
D and B, which are identical or different, are each —O—, —S—, —NH—, —CH₂—, —CH═CH—, or —N(C₁-C₄-alkyl)-;
R¹⁶ is hydrogen, hydroxy, —C₁-C₄-alkyl, —C₁-C₄-alkyloxy, —C₁-C₄-alkylene-halogen, —O—C₁-C₄-alkylene-halogen, —C₁-C₄-alkylene-OH, —CF₃, —CHF₂, —C₁-C₄-alkylene-C₁-C₄-alkyloxy, —O—COC₁-C₄-alkyl, —O—COC₁-C₄-alkylene-halogen, —C₁-C₄-alkylene-C₃-C₆-cycloalkyl, —O—COCF₃, or halogen;
R^{1″} and R^{2″}, which are identical or different, are each —C₁-C₅-alkyl optionally substituted by —C₃-C₆-cycloalkyl, hydroxy, or halogen, or R^{1″} and R^{2″} together are a —C₃-C₅-alkylene bridge;
R¹⁷, R¹⁸, R^{17′}, and R^{18′}, which are identical or different, are each hydrogen, C₁-C₄-alkyl, C₁-C₄-alkyloxy, hydroxy, —CF₃, —CHF₂, —CN, —NO₂, or halogen;
R^{x} and R^{x′}, which are identical or different, are each hydrogen, C₁-C₄-alkyl, C₁-C₄-alkyloxy, hydroxy, —CF₃, —CHF₂, —CN, —NO₂, or halogen, or R^{x} and R^{x′} together are a single bond or a bridging group selected from —O—, —S—, —NH—, —CH₂—, —CH₂—CH₂—, —N(C₁-C₄-alkyl)-, —CH(C₁-C₄-alkyl)-, and —C(C₁-C₄-alkyl)₂-,
or an enantiomer, racemate, hydrate, or mixture thereof.

24. The pharmaceutical composition of claim 23, wherein X⁻ is fluoride, chloride, bromide, iodide, sulfate, phosphate, methanesulfonate, nitrate, maleate, acetate, citrate, fumarate, tartrate, oxalate, succinate, benzoate, or p-toluenesulfonate, or an enantiomer, racemate, hydrate, or mixture thereof.

25. The pharmaceutical composition of claim 1, wherein the anticholinergic is a compound of formula 1g 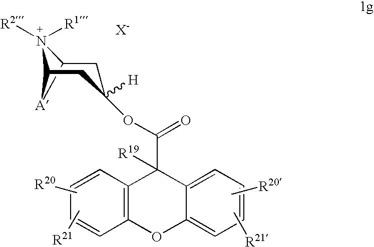
wherein:
X⁻ is an anion with a single negative charge; 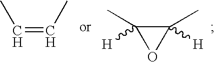
A is
R¹⁹ is hydroxy, methyl, hydroxymethyl, ethyl, —CF₃, —CHF₂, or fluorine;
R^{1′″} and R^{2′″}, which are identical or different, are each C₁-C₅-alkyl optionally substituted by C₃-C₆-cycloalkyl, hydroxy, or halogen, or R^{1′″}′and R^{2′″} together are a —C₃-C₅-alkylene-bridge; and
R²⁰, R²¹, R^{20′}, and R^{21′}, which are identical or different, are each hydrogen, —C₁-C₄-alkyl, —C₁-C₄-alkyloxy, hydroxy, —CF₃, —CHF₂, —CN, —NO₂, or halogen,
or an enantiomer, racemate, hydrate, or mixture thereof.

26. The pharmaceutical composition of claim 25, wherein X⁻ is fluoride, chloride, bromide, iodide, sulfate, phosphate, methanesulfonate, nitrate, maleate, acetate, citrate, fumarate, tartrate, oxalate, succinate, benzoate, or p-toluenesulfonate, or an enantiomer, racemate, hydrate, or mixture thereof.

27. The pharmaceutical composition according to one of claims 1, 2, 3, 4, 5, 8, 10, 12, 15, 1, 7, 19, 21, 23, or 25, wherein the pharmaceutical composition is suitable for inhalation.

28. The pharmaceutical composition according to claim 27, wherein the pharmaceutical composition is an inhalable powder, a propellant-containing metering aerosol, or a propellant-free inhalable solution or suspension.

29. The pharmaceutical composition according to claim 1, wherein the pharmaceutical composition further comprises a suitable physiologically acceptable excipient selected from the group consisting of: monosaccharides, disaccharides, oligo- and polysaccharides, polyalcohols, and salts.

30. The pharmaceutical composition of claim 29, wherein the excipient has a maximum average particle size of up to 250 μm.

31. The pharmaceutical composition of claim 30, wherein the excipient has a maximum average particle size of between 10 μm and 150 μm.

32. The pharmaceutical composition of claim 31, wherein the excipient has a maximum average particle size of between 10 μm and 150 μm.

33. A capsule containing a pharmaceutical composition according to one of claims 1, 2, 3, 4, 5, 8, 10, 12, 15, 17, 19, 21, 23, or 25 in the form of an inhalable powder.

34. A capsule containing a pharmaceutical composition according to claim 27 in the form of an inhalable powder.

35. A pharmaceutical composition according to claim 1, wherein the pharmaceutical composition is a propellant-containing inhalable aerosol and the anticholinergic and the betamimetic are in dissolved or dispersed form.

36. The pharmaceutical composition according to claim 35, wherein the propellant-containing inhalable aerosol comprises a propellant gas selected from hydrocarbons and halohydrocarbons.

37. The pharmaceutical composition according to claim 35, wherein the propellant-containing inhalable aerosol comprises a propellant gas selected from the group consisting of: n-propane; n-butane; isobutane; and chlorinated and/or fluorinated derivatives of methane, ethane, propane, butane, cyclopropane, and cyclobutane.

38. The pharmaceutical composition according to claim 36, wherein the propellant gas is TG134a, TG227, or a mixture thereof.

39. The pharmaceutical composition according to claim 35, further comprising at least one of a cosolvent, stabilizer, surfactant, antioxidant, lubricant, or means for adjusting the pH of the composition.

40. The pharmaceutical composition according to one of claims 36 to 38, further comprising at least one of a cosolvent, stabilizer, surfactant, antioxidant, lubricant, or means for adjusting the pH of the composition.

41. The pharmaceutical composition according to claim 35, wherein the amount of the anticholinergic and the betamimetic is up to 5 wt. % of the pharmaceutical composition.

42. A pharmaceutical composition according to claim 1, wherein the pharmaceutical composition is propellant-free inhalable solution or suspension that further comprises a solvent selected from water, ethanol, or a mixture of water and ethanol.

43. The pharmaceutical composition according to claim 42, wherein the pH is between 2 and 7.

44. The pharmaceutical composition according to claim 43, wherein the pH is between 2 and 5.

45. The pharmaceutical composition according to claim 42, wherein the pH of the pharmaceutical composition is adjusted by means of one or more acids selected from the group consisting of: hydrochloric acid, hydrobromic acid, nitric acid, sulfuric acid, ascorbic acid, citric acid, malic acid, tartaric acid, maleic acid, succinic acid, fumaric acid, acetic acid, formic acid, and propionic acid.

46. The pharmaceutical composition according to claim 42, further comprising other cosolvents or excipients.

47. The pharmaceutical composition according to claim 45, further comprising other cosolvents or excipients.

48. The pharmaceutical composition according to claim 46, wherein the cosolvent is selected from the group consisting of alcohols, glycols, polyoxyethylene alcohols, and polyoxyethylene fatty acid esters.

49. The pharmaceutical composition according to claim 46, wherein the cosolvent is selected from the group consisting of: isopropyl alcohol, propylene glycol, polyethylene glycol, polypropylene glycol, glycol ether, and glycerol.

50. The pharmaceutical composition according to claim 46, wherein the excipient is selected from the group consisting of: surfactants, stabilizers, complexing agents, antioxidants, preservatives, flavorings, pharmacologically acceptable salts, and vitamins.

51. The pharmaceutical composition according to claim 50, wherein the excipient is selected from the group consisting of: edetic acid, a salt of edetic acid, ascorbic acid, vitamin A, vitamin E, tocopherols, cetyl pyridinium chloride, benzalkonium chloride, benzoic acid, and benzoate salts.

52. A method of treating asthma, COPD, or other inflammatory or obstructive respiratory complaints in a patient in need of such treatment, the method comprising administering to the patient a therapeutically effective amount of the pharmaceutical composition according to one of claims 1, 2, 8, 10, 1, 2, 1, 5, 1, 7, 19, 21, 23, or 25.

53. A kit comprising one or more unit dosage containers containing a pharmaceutical composition, each unit dosage container containing a pharmaceutical composition comprising:
(a) a betamimetic of formula 2 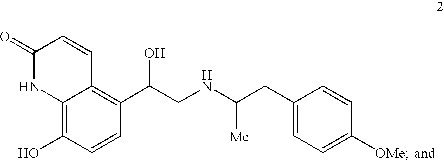
and
(b) an anticholinergic,
each optionally together with a pharmaceutically acceptable excipient,
the anticholinergic and the betamimetic optionally in the form of their enantiomers, mixtures of their enantiomers, their racemates, their solvates, or their hydrates.

54. The kit according to claim 53, further comprising instructions with directions for using the kit.

55. A kit comprising:
(a) a first container containing a first pharmaceutical formulation comprising a betamimetic of formula 2 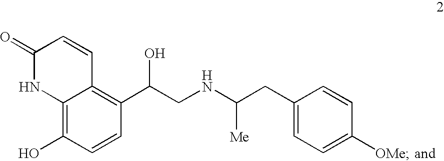
10 and
(b) a second container containing a second pharmaceutical formulation comprising an anticholinergic,
each container each optionally further containing a pharmaceutically acceptable excipient,
the anticholinergic and the betamimetic optionally in the form of their enantiomers, mixtures of their enantiomers, their racemates, their solvates, or their hydrates.

56. The kit according to claim 55, further comprising instructions with directions for using the kit.
